SEQUENCE LISTING (4 sequences)

SEQ ID NO: 1 - RNA, 20 nt, synthetic construct
  source 1..20: mol_type = other RNA
gattaaataggcacatgtgt

SEQ ID NO: 2 - RNA, 20 nt, synthetic construct
  source 1..20: mol_type = other RNA
aaaattattttagattaaat

SEQ ID NO: 3 - RNA, 20 nt, synthetic construct
  source 1..20: mol_type = other RNA
gacaaggttcatatttgtat

SEQ ID NO: 4 - RNA, 20 nt, synthetic construct
  source 1..20: mol_type = other RNA
agcagtgtgtttggagagca

(19) United States
(12) Patent Application Publication
(10) Pub. No.: US 2025/0075207 A1
(43) Pub. Date: Mar. 6, 2025

(54) TISSUE SPECIFIC METHODS AND COMPOSITIONS FOR GENE EDITING

(71) Applicant: Helex Inc., Lewes, DE (US)

(72) Inventors: Poulami Chaudhuri, Hyderabad (IN); Rohini Kalvakuntla, Hyderabad (IN); Neha Gaur, Hyderabad (IN); Rakesh Kappagantu, Hyderabad (IN); Aniruddha Nishtala, Hyderabad (IN); Harsha Rohira, Hyderabad (IN)

(21) Appl. No.: 18/953,729

(22) Filed: Nov. 20, 2024

Related U.S. Application Data
(63) Continuation of application No. PCT/US2023/022978, filed on May 19, 2023.
(60) Provisional application No. 63/368,936, filed on Jul. 20, 2022.

(30) Foreign Application Priority Data
May 20, 2022 (IN) 202221029160

(51) Int. Cl. C12N 15/11; C12N 9/22

(52) U.S. Cl. CPC C12N 15/11; C12N 9/22; C12N 2310/20; C12N 2330/30

(57) ABSTRACT

Methods are provided for making a guide RNA (gRNA) suitable for use within a RNA-guided endonuclease complex, wherein the gRNA comprises a nucleic acid sequence that is configured to hybridise with a target nucleic acid sequence within the genome of a cell, and wherein the target nucleic acid sequence is characterised as being comprised within a locus that is active within a specific cell type. Also provided are RNA-guided endonuclease complexes and cell or tissue type selective or preferential gRNAs.

### CROSS-REFERENCE TO RELATED APPLICATIONS

This application is a continuation of PCT/US2023/022978, filed May 19, 2023; which claims the benefit of Indian patent application Ser. No. 20/222,1029160, filed May 20, 2022, and U.S. Provisional Application No. 63/368,936, filed Jul. 20, 2022. The contents of the above-identified applications are incorporated herein by reference in their entireties.

### SEQUENCE LISTING

This application contains an ST.26 compliant Sequence Listing, which was submitted in xml format via Patent Center and is hereby incorporated by reference in its entirety. The .xml copy, created on May 18, 2023 is named “P6894WO_seq_lst.xml” and is 4300 bytes in size.

### BACKGROUND OF THE INVENTION

RNA-targeted endonucleases such as the clustered regularly interspaced short palindromic repeat (CRISPR)/CRISPR-associated system (Cas) gene-editing system represent a promising tool for therapeutic genome manipulation. However, to date the low intracellular delivery efficiency and poor cell/tissue type specificity has severely compromised the potential for this technology in clinical applications.

The main challenges associated with translating CRISPR based therapies to the clinic are due to their associated off-target effects, which lead to unwanted genomic changes which can have serious consequences for the patient (Hoijer et al. (2022) Nature communications, 13(1), pp. 1-10). Also, the inability to efficiently target CRISPR/Cas editing system to the tissue of interest without any unwanted biodistribution to other tissues leads to undesirable gene editing. This remains a particular challenge limiting the use of systemic delivery technologies. Although there is a lot of innovation around tissue specific vector design and use of regulatory elements to confer tissue specific Cas expression, there are still many unresolved issues associated with these approaches such as lack of complete control on the biodistribution of the designed vector or the uncontrolled expression of Cas enzyme and the difficulties associated with payload capacities, raising safety concerns in the long term.

Current methods for tissue specific gene editing include vector targeting. However, reliance on vector targeting exhibits the following disadvantages:
1. Vector designing for tissue specificity: using adeno-associated viral (AAV) and and non-viral (LNPs, Peptide based nanoparticles), the industry has not yet achieved 100% targeted delivery. AAVs have better tissue targeting ability, however several natural AAVs have the ability to cross physiological barriers to transduce non target tissues.
2. Capacity issues: Non-viral vectors such as lipidoid nanoparticles (LNPs) are able to carry larger payloads, but are non-specifically targeted. Under in vivo conditions, they show wide biodistribution and tend to congregate in the liver. Various ligands, aptamers etc. can be used to make LNPs somewhat more specific but can increase immunogenicity. Whilst AAVs have more tissue specific targetability, they are severely restricted by size of the payload, with a typical AAV payload limited to less than 5 kb of DNA.

The use of non-viral delivery methods is preferred for in vivo editing, and the challenge of mitigating gene editing in off-target cells and tissues is still of growing concern.

Hence there is a need to improve the cell and tissue specific targeting of RNA-targeted endonucleases such that conventional delivery technologies such as LNPs or AAVs can be used effectively without risk of off-target effects.

These and other uses, features and advantages of the invention should be apparent to those skilled in the art from the teachings provided herein.

### SUMMARY OF THE INVENTION

The present inventors provide for novel methods to design and synthesise cell and tissue specific guide RNAs (gRNAs) to enhance safety of in vivo RNA-guided endonuclease complexes, such as those used in CRISPR/Cas based gene-editing therapies. The advantages of the invention include production of novel gRNAs that are highly cell and tissue specific allowing for gene editing only in the intended cell types with minimal to no editing in unintended cell-types or tissues.

Accordingly, in a first aspect the invention provides a method for making a guide RNA (gRNA), wherein the gRNA comprises a nucleic acid sequence that is configured to hybridise with a target nucleic acid sequence within the genome of a cell, wherein the target nucleic acid sequence is characterised as being comprised within a locus that is active within a specific cell type, the method comprising the steps of:
i. identifying one or more candidate nucleic acid sequences that are unique to a specific cell type as compared to a control cell that is selected from a different cell type, thereby identilying tissue specific candidate nucleic acid sequences;
ii. identifying a subset of the candidate nucleic acid sequences of (i) that are comprised within an accessible chromatin region of the genome within the specific cell type; and
iii. synthesising a gRNA that hybridises to one or more of the tissue specific candidate nucleic acid sequences identified in (ii).

In an embodiment the one or more candidate nucleic acid sequences comprise or are adjacent to a protospacer adjacent motif (PAM) sequence.

In an embodiment the accessible chromatin region of the genome is comprised within a region of euchromatin.

In an embodiment the accessible chromatin region of the genome is comprised within a gene.

In an embodiment the gene is predominantly expressed or uniquely regulated only within the specific cell type.

In an embodiment the accessible chromatin region of the genome is fully or partially comprised within an untranslated region of the gene.

In an embodiment the tissue specific candidate nucleic acid sequences are defined as comprising at least one tissue specific gene expression control sequence.

In an embodiment at least one tissue specific gene expression control sequence is selected from the group consisting of: a promoter; an enhancer; a silencer; an insulator; an miRNA; an IncRNA; a transcription factor; and a transcription factor binding sequence.

In an embodiment the specific cell type is selected from the group consisting of: muscle; liver; central nervous system (CNS); brain; breast; endothelium; pancreas; esophagus; colon; gastrointestinal organs; kidney; lung; spleen; skin; heart; thyroid; lymphatic tissue; cardiovascular; eye; bone marrow; blood; connective tissue; bladder; reproductive organs; and placenta.

In an embodiment the specific cell type comprises a diseased cell type.

In an embodiment the diseased cell type is caused by an intracellular pathogen.

In an embodiment the diseased cell type is selected from a pre-neoplastic or a neoplastic cell type and wherein neoplastic cell type is selected from the group consisting of: a primary tumour cell; a secondary tumour cell; a metastatic tumour cell; and a cancer stem cell.

In an embodiment the gRNA is a single gRNA (sgRNA).

In an embodiment the gRNA or sgRNA is selected based on optimal on-target cleavage and minimum off-target activity predictions.

A second aspect of the invention provides a nucleic acid library that comprises a plurality of nucleic acid sequences that encode a plurality of gRNAs identified via any of the methods described herein.

A third aspect of the invention provides an engineered guide RNA (gRNA) capable of forming a complex with a CRISPR-Cas endonuclease protein and directing the CRISPR-Cas complex to a tissue or cell type specific target sequence within a locus of a cell; wherein the gRNA comprises a sequence that hybridises with the tissue or cell type specific target sequence, and wherein the gRNA is synthesised by any of the methods as described herein.

A fourth aspect provides a method for making a guide RNA (gRNA), wherein the gRNA comprises a nucleic acid sequence that is configured to hybridise with a target nucleic acid sequence within a locus in the genome of a target cell type, wherein the target nucleic acid sequence is characterised as being comprised within a locus that is epigenetically accessible within the target cell type, the method comprising the steps of:
i. identifying one or more candidate nucleic acid sequences that are comprised within the locus and that are unique to the target cell type as compared to a control cell that is selected from a different cell type, thereby identifying one or more tissue specific candidate nucleic acid sequences;
ii. identifying a subset of the candidate nucleic acid sequences of (i) that are comprised within an accessible chromatin region of the genome within the target cell type; and
iii. synthesising a gRNA that hybridises to one or more of the tissue specific candidate nucleic acid sequences identified in (ii).

In an embodiment the one or more candidate nucleic acid sequences comprise or are adjacent to a protospacer adjacent motif (PAM) sequence.

In an embodiment the locus that is epigenetically accessible is comprised within a region of euchromatin.

In an embodiment the locus that is epigenetically accessible is comprised within a gene.

In an embodiment the gene is predominantly expressed or uniquely regulated only within the target cell type.

In an embodiment the locus that is epigenetically accessible is fully or partially comprised within an untranslated region of the gene.

In an embodiment the locus comprises a specific gene expression control sequence selected from the group consisting of: a promoter; an enhancer; a silencer; an insulator; and a transcription factor binding sequence.

In an embodiment the target cell type is selected from the group consisting of: muscle; liver; central nervous system (CNS); brain; breast; endothelium; pancreas; esophagus; colon; gastrointestinal organs; kidney; lung; spleen; skin; heart; thyroid; lymphatic tissue; cardiovascular; eye; bone marrow; blood; connective tissue; bladder; reproductive organs; and placenta.

In an embodiment the specific cell type comprises a diseased cell type.

In an embodiment the diseased cell type is caused by an intracellular pathogen.

In an embodiment the diseased cell type is selected from a pre-neoplastic or a neoplastic cell type and wherein neoplastic cell type is selected from the group consisting of: a primary tumour cell; a secondary tumour cell; a metastatic tumour cell; and a cancer stem cell.

In an embodiment the gRNA is a single gRNA (sgRNA).

In an embodiment the gRNA or sgRNA is selected based on optimal on-target cleavage and minimum off-target activity predictions.

A fifth aspect of the invention provides a CRISPR-Cas complex that comprises an engineered guide RNA (gRNA) in a complex with a CRISPR-Cas endonuclease protein, wherein the gRNA is capable of directing the complex to a tissue or cell type specific target sequence within a locus of a cell; wherein the gRNA comprises a sequence that hybridises with the tissue or cell type specific target sequence, and wherein the gRNA is synthesised by any of the methods as described herein.

In an embodiment the CRISPR-Cas endonuclease is selected from the group consisting of: Cas9; Cpf1; c2cl; C2c2; Casl3; c2c3; Cas1; Cas1B; Cas2; Cas3; Cas4; Cas5; Cas5e (CasD); Cas6; Cas6e; Cas6f; Cas7; Cas8; Cas8a; Cas8a1; Cas8a2; Cas8b; Cas8c; Csnl; Csxl2; Cas9; Cas10; Cas10d; Cas12a; Cas12b; Cas12c; Cas12d; Cas12e; Cas13a; Cas13b; Cas13c; Cas13d; CasF; CasG; CasH; Csyl; Csy2; Csy3; Csel (CasA); Cse2 (CasB); Cse3 (CasE); Cse4 (CasC); Cse5; Cscl; Csc2; Csa5; Csn2; Csm2; Csm3; Csm4; Csm5; Csm6; Cmrl; Cmr3; Cmr4; Cmr5; Cmr6; Csbl; Csb2; Csb3; Csx17; Csxl4; CsxIO; Csx16; CsaX; Csx3; Csxl; Csx15; Csfl; Csf2; Csf3; Csf4; and Cul966, or a derivative thereof; a variant thereof; and a fragment thereof.

In an embodiment the CRISPR-Cas endonuclease is a Cas9 or a derivative thereof; a variant thereof; and a fragment thereof.

In an embodiment the CRISPR-Cas endonuclease is a Cpf1 or a derivative thereof; a variant thereof; and a fragment thereof.

A sixth aspect of the invention provides for a gRNA comprising a sequence selected from any one of the group consisting of SEQ ID NOs: 1-4. In embodiments, therapeutic compositions comprising any one of the sequences of SEQ ID NOs: 1-4 are provided, including therapeutic compositions that comprise a CRISPR-Cas endonuclease

A seventh aspect of the invention provides a pharmaceutical composition comprising an engineered guide RNA (gRNA) capable of forming a complex with a CRISPR-Cas effector protein and directing the CRISPR-Cas complex to a tissue or cell type specific target sequence within a locus of a cell; wherein the gRNA comprises a sequence of SEQ ID NO: 1 or 2. Suitably the composition is for use in a method of treating a disease selected from: hormone-dependent forms of cancer, breast cancer, prostate cancer, endometrial cancer, premenstrual syndrome, endometriosis, catamenial epilepsy or a depressive disorder.

An eighth aspect of the invention provides a pharmaceutical composition comprising an engineered guide RNA (gRNA) capable of forming a complex with a CRISPR-Cas effector protein and directing the CRISPR-Cas complex to a tissue or cell type specific target sequence within a locus of a cell; wherein the gRNA comprises a sequence of SEQ ID NO: 3. Suitably, the composition is for use in a method of treating amyloid TTR (ATTR) amyloidosis.

A ninth aspect of the invention provides a pharmaceutical composition comprising an engineered guide RNA (gRNA) capable of forming a complex with a CRISPR-Cas effector protein and directing the CRISPR-Cas complex to a tissue or cell type specific target sequence within a locus of a cell; wherein the gRNA comprises a sequence of SEQ ID NO: 4. Suitably the composition is for use in a method of treating a human lipoprotein metabolism disorder.

Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

One or more embodiments of the invention will now be described, by way of example only, with reference to the accompanying drawings, in which:

FIG. 1 shows a schematic of the accessibility of chromatin for gene editing in diseased v healthy tissue.

FIG. 2 shows a schematic of the bioinformatic whole exome sequence (WES) pipeline.

FIG. 3 shows a schematic of the transcriptomic RNA-seq pipeline.

FIG. 4 shows a methodology for determining the specificity of gene editing in target cell line v non-target cell line.

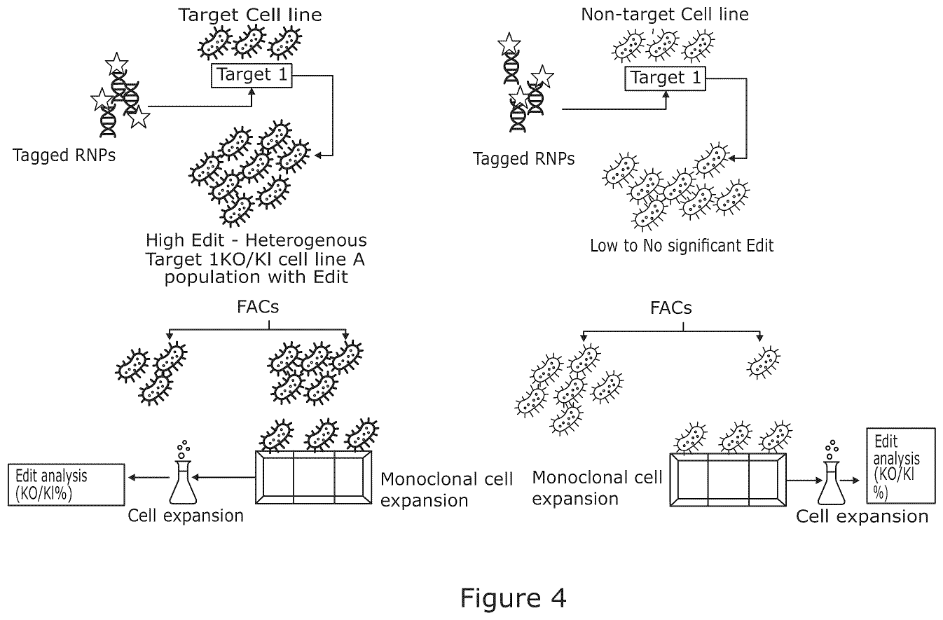

FIG. 5 shows a schematic of a method for designing liver specific gene editing apparatus and determining efficiency of edit in liver cells v non-liver cells.

FIG. 6A shows the results of an agarose gel electrophoresis for T7E1 assay of AKR1C4 in target versus non-target cells.

FIG. 6B is a graph of results in FIG. 6A showing gene-edit percentages calculated per T7E1 gel using Gene-analyser.

FIG. 7A shows the results of an agarose gel electrophoresis for T7E1 assay of TTR in target versus non-target cells.

FIG. 7B is a graph showing results of selective editing in the T7E1 assay of TTR in target liver hepatocytes versus non-target cells of FIG. 7A, gene-edit percentages calculated per a T7E1 gel using Gene-analyser.

FIG. 8A shows the results of an agarose gel electrophoresis for T7E1 assay of ANGPTL3 in target versus non-target cells.

FIG. 8B is a graph showing results of preferential editing in a T7E1 assay of ANGPTL3 in target liver hepatocytes versus non-target cells of FIG. 8A, gene-edit percentages calculated per a T7E1 gel using Gene-analyser.

FIG. 9A shows the results of an agarose gel electrophoresis for T7E1 assay of KLKB1 in target versus non-target cells.

FIG. 9B is a graph showing results of selective editing in a T7E1 assay of KLKB1 in liver hepatocytes versus colon cells of FIG. 9A, gene-edit percentages calculated per a T7E1 gel using Gene-analyser.

### DETAILED DESCRIPTION OF THE INVENTION

Unless otherwise indicated, the practice of the present invention employs conventional techniques of chemistry, molecular biology, microbiology, recombinant DNA technology, and chemical methods, which are within the capabilities of a person of ordinary skill in the art. Such techniques are also explained in the literature, for example, M. R. Green, J. Sambrook, 2012, Molecular Cloning: A Laboratory Manual, Fourth Edition, Books 1-3, Cold Spring Harbor Laboratory Press, Cold Spring Harbor, NY; Ausubel, F. M. et al. (Current Protocols in Molecular Biology, John Wiley & Sons, Online ISSN: 1934-3647); B. Roe, J. Crabtree, and A. Kahn, 1996, DNA Isolation and Sequencing: Essential Techniques, John Wiley & Sons; J. M. Polak and James O'D. McGee, 1990, In Situ Hybridisation: Principles and Practice, Oxford University Press; M. J. Gait (Editor), 1984, Oligonucleotide Synthesis: A Practical Approach, IRL Press; and D. M. J. Lilley and J. E. Dahlberg, 1992, Methods of Enzymology: DNA Structure Part A: Synthesis and Physical Analysis of DNA Methods in Enzymology, Academic Press; Synthetic Biology, Part A, Methods in Enzymology, Edited by Chris Voigt, Volume 497, pages 2-662 (2011); Synthetic Biology, Part B, Computer Aided Design and DNA Assembly, Methods in Enzymology, Edited by Christopher Voigt, Volume 498, Pages 2-500 (2011); RNA Interference, Methods in Enzymology, David R. Engelke, and John J. Rossi, Volume 392, Pages 1-454 (2005). All references cited herein are incorporated by reference in their entirety. Unless otherwise defined, all technical and scientific terms used herein have the same meaning as commonly understood by one of ordinary skill in the art to which this invention belongs.

As used herein, the term ‘comprising’ means any of the recited elements are necessarily included and other elements may optionally be included as well. ‘Consisting essentially of’ means any recited elements are necessarily included, elements that would materially affect the basic and novel characteristics of the listed elements are excluded, and other elements may optionally be included. ‘Consisting of’ means that all elements other than those listed are excluded. Embodiments defined by each of these terms are within the scope of this invention.

The term ‘operably linked’ refers to the joining of distinct DNA molecules, or DNA sequences, to produce a functional transcriptional unit. When applied to DNA sequences, for example in an expression vector or a recombinantly modified gene construct, it indicates that the sequences are arranged, or juxtaposed, so that they function cooperatively in order to achieve their intended purposes, e.g. a promoter sequence allows for initiation of transcription that proceeds through a linked coding sequence as far as a termination sequence.

A ‘polynucleotide’ is a single or double stranded covalently-linked sequence of nucleotides in which the 3′ and 5′ ends on each nucleotide are joined by phosphodiester bonds. The polynucleotide may be made up of deoxyribonucleotide bases or ribonucleotide bases. Polynucleotides include DNA and RNA, and may be manufactured synthetically in vitro or isolated from natural sources. Sizes of polynucleotides are typically expressed as the number of base pairs (bp) for double stranded polynucleotides, or in the case of single stranded polynucleotides as the number of nucleotides (nt). One thousand bp or nt equal a kilobase (kb). Polynucleotides of less than around 40 nucleotides in length are typically called “oligonucleotides”. The term further includes known types of chemical modifications, for example, labels which are known in the art, methylation, caps, substitution of one or more of the naturally occurring nucleotides with nucleotide modifications such as pseudouridine, or those with uncharged linkages (e.g., phosphorothioates, phosphorodithioates, etc.), those containing nucleotide analogues (e.g., peptide nucleic acids and locked nucleic acids), as well as unmodified forms of the polynucleotide. Hence, non-naturally occurring nucleotides and/or nucleotide analogues may be modified at the ribose, phosphate, and/or base moiety. In an embodiment of the invention, a guide nucleic acid comprises ribonucleotides and non-ribonucleotides. In one such embodiment, a nucleic acid guide molecule comprises one or more ribonucleotides and one or more deoxyribonucleotides. In another embodiment of the invention, the nucleic acid guide comprises one or more non-naturally occurring nucleotide or nucleotide analogues such as a nucleotide with phosphorothioate linkage, a locked nucleic acid (LNA) nucleotides comprising a methylene bridge between the 2′ and 4′ carbons of the ribose ring, or bridged nucleic acids (BNA). Further examples of modified nucleotides include 2′-O-methyl analogues, 2′-deoxy analogues, or 2′-fluoro analogues. Further examples of modified bases include, but are not limited to, 2-aminopurine, 5-bromo-uridine, pseudouridine, inosine, 7-methylguanosine. Examples of guide RNA chemical modifications include, without limitation, incorporation of 2′-O-methyl (M), 2′-O-methyl 3′phosphorothioate (MS), S-constrained ethyl (cEt), or 2′-O-methyl 3′thioPACE (MSP) at one or more terminal nucleotides.

When expression is described as being “predominantly” in a given tissue, this indicates that the gene's mRNAs levels are highest in this tissue as compared to the other tissues in which it was measured.

As used herein, the term “gene” refers to any nucleotide sequence encoding a known or putative gene product. The gene includes the regulatory regions, such as the promoter and enhancer regions, the transcribed regions, which include the coding regions, and other functional sequence regions.

As used herein, the terms ‘3′’ (‘3 prime’) and ‘5′’ (‘5 prime’) take their usual meanings in the art, i.e. to distinguish the ends or directionality within linear polynucleotide molecules. A polynucleotide has a 5′ and a 3′ end and polynucleotide sequences are conventionally written in a 5′ to 3′ direction. The 5′ end is suitably considered to be upstream of the 3′ end of a polynucleotide sequence. Hence, sequence referred to as upstream of a given reference point in a gene, such as the transcription start codon of an open reading frame (ORF), is sequence that is 5′ to the reference point. Likewise sequence denoted as downstream is 3′ to the reference point.

The term ‘gene expression control sequence’ comprises regulatory sequences, sometimes referred to as a cis-regulatory element (CRE) and includes promoters, ribosome binding sites, enhancers, silencers and insulators and other control elements which regulate transcription of a gene or translation of a resultant mRNA. In particular embodiments of the invention, the gene expression control sequences confer tissue or cell-type specificity that assist in determining the phenotype of the cell. Gene expression control sequences may also contribute to regulation of gene expression levels. For example, the expression level of a particular gene can be considered as the amount of mRNA and/or polypeptide produced from that particular gene. Gene expression levels can refer to an absolute (e.g., molar or gram-quantity) abundance of mRNA or polypeptide, or a relative (e.g., the amount relative to a standard, reference, calibration, or to another gene expression level).

Cell-type specificity refers to the observable characteristics or traits of a particular cell, such as its morphology, development, biochemical or physiological properties, phenology, or behaviour. Cell-type specificity also refers to the epigenetic characteristics of a particular cell. The cell-type may refer to the ‘phenotype’ of the cell and results primarily from the expression of the genes within the cell as well as any influence from external/environmental factors, such as disease pathogens or physical stresses (e.g. hypoxia, hypo-or hyperthermia and/or dehydration). In specific embodiments, a genetic regulatory element that confers cell or tissue type specificity may be defined as a tissue-specific regulatory element. Such tissue-specific regulators may include promoter sequences that direct gene expression primarily in a desired tissue of interest. They may also include enhancers, insulators, mRNAs, IncRNAs, other transcription factors, transcription factor binding sites, etc. Tissues or cells may be comprised within organ systems within the body, such as but not limited to those selected from the group consisting of: muscle; liver; central nervous system (CNS); brain; breast; endothelium; pancreas; esophagus; colon; gastrointestinal tract; kidney; lung; spleen; skin; heart; thyroid; lymphatic tissue; cardiovascular; eye; bone marrow; blood; connective tissue; bladder; reproductive organs; and placenta. Within each organ system there are multiple tissue and cellular subtypes as well as less differentiated cells, e.g. precursor and stem cells. Hence, as used herein, the term ‘organ’ is synonymous with an ‘organ system’ and refers to a combination of tissues and/or cell types that may be compartmentalised within the body of a subject to provide a biological function, such as a physiological, anatomical, homeostatic or endocrine function. Suitably, organs or organ systems may mean a vascularized internal organ, such as a liver or pancreas. Typically organs comprise at least two tissue types, and/or a plurality of cell types that exhibit a phenotype characteristic of the organ.

In addition, many organs may comprise so-called healthy or non-aberrant pathology as well as non-healthy or diseased cells. The term ‘diseased’ as used herein, as in ‘diseased cells’ and/or diseased tissue' indicates tissues and organs (or parts thereof) and cells which exhibit an aberrant, non-healthy or disease pathology. For instance, diseased cells may be infected with a virus, bacterium, prion or eukaryotic parasite; may comprise deleterious mutations; and/or may be cancerous, precancerous, tumoural or neoplastic. In certain instances disease cells may be pathologically normal but comprise an altered intra-cellular miRNA environment that represents a precursor state to disease. Diseased tissues may comprise healthy tissues that have been infiltrated by diseased cells from another organ or organ system. By way of example, many inflammatory diseases comprise pathologies where otherwise healthy organs are subjected to infiltration with immune cells such as T cells and neutrophils. By way of a further example, organs and tissues subjected to stenotic or cirrhotic lesions may comprise both healthy and diseased cells in close proximity.

The term ‘cancer’ as used herein refers to neoplasms in tissue, including malignant tumours which may be primary cancer starting in a particular tissue, or secondary cancer having spread by metastasis from elsewhere. The terms cancer, neoplasm and malignant tumours are used interchangeably herein. Cancer may denote a tissue or a cell located within a neoplasm or with properties associated with a neoplasm. Neoplasms typically possess characteristics that differentiate them from normal tissue and normal cells. Among such characteristics are included, but not limited to: a degree of anaplasia, changes in morphology, irregularity of shape, reduced cell adhesiveness, the ability to metastasize, and increased cell proliferation. Terms pertaining to and often synonymous with ‘cancer’ include sarcoma, carcinoma, malignant tumour, epithelioma, leukaemia, lymphoma, transformation, neoplasm and the like. As used herein, the term ‘cancer’ includes premalignant, and/or precancerous tumours, as well as malignant cancers.

The term ‘healthy’ as used herein, as in ‘healthy cells’ and/or ‘healthy tissue’ indicates tissues and organs (or parts thereof) and cells which are not themselves diseased and approximate to a typically normal functioning phenotype. It can be appreciated that in the context of the invention the term ‘healthy’ is relative, as, for example, non-neoplastic cells in a tissue affected by tumours may well not be entirely healthy in an absolute sense. Therefore ‘non-healthy cells’ is used mean cells which are not themselves neoplastic, cancerous or pre-cancerous but which may be cirrhotic, inflamed, or infected, or otherwise diseased for example. Similarly, ‘healthy or non-healthy tissue’ is used to mean tissue, or parts thereof, without tumours, neoplastic, cancerous or pre-cancerous cells; or other diseases as mentioned above; regardless of overall health. For instance, in the context of an organ comprising cancerous and fibrotic tissue, cells comprised within the fibrotic tissue may be thought of as relatively ‘healthy’ compared to the cancerous tissue.

The term ‘promoter’ as used herein denotes a genetic regulatory element in a DNA sequence to which an RNA polymerase will bind and initiate transcription of the DNA. Promoters play a crucial role in gene expression by providing a binding site for RNA polymerases. When RNA polymerase binds to the promoter region, it initiates the process of transcription. Promoters are typically, but not always, located in the 5′ non-coding regions of genes. The 5′ region refers to the upstream region of a gene, meaning it precedes the actual coding sequence of the gene often denoted by an ATG start codon (e.g. prior to the first exon). Non-coding regions are segments of DNA that do not directly contribute to the formation of a polypeptide or other gene product. These regions can contain various regulatory elements, including the promoter. The primary function of a promoter sequence is to provide a recognition site for RNA polymerase and other transcriptional regulatory proteins, allowing them to interact with the DNA and initiate the transcription process. The binding of RNA polymerase to the promoter region marks the starting point for the assembly of the transcriptional machinery, which ultimately leads to the synthesis of an RNA molecule known as the primary transcript or pre-mRNA. Consequently, promoters are highly diverse in terms of their sequence and structure. They contain specific DNA motifs and sequences that are recognized by transcription factors that further regulate gene expression. Transcription factors can either enhance or Inhibit the binding of RNA polymerase to the promoter, thereby influencing the level of gene transcription, often in a cell-type or tissue specific manner.

The term ‘enhancer’ as used herein denotes a genetic regulatory element in a DNA sequence that, when bound by one or more transcription factors, enhances the transcription of an associated gene. Enhancers play a pivotal role in gene expression by regulating the transcription of an associated gene or set of genes within a locus. When an enhancer is bound by one or more transcription factors, it enhances the rate of transcription. Enhancers are typically located at varying distances from the gene(s) they regulate. They can be found either upstream (upstream enhancers) or downstream (downstream enhancers) of the gene(s), and sometimes even within introns within the gene itself. Unlike promoters, enhancers are not necessarily orientation-specific and can function regardless of their orientation relative to the gene. A key function of an enhancer is to provide a binding site for transcription factors and regulatory complexes. When specific transcription factors recognize and bind to the enhancer, they can facilitate the assembly of the transcriptional machinery at the promoter region of the associated gene. This recruitment and interaction of transcription factors at the enhancer and promoter regions enable efficient initiation and regulation of gene transcription. Enhancers exhibit remarkable flexibility and can act over long distances. They can interact with the promoter region of the target gene through three-dimensional looping of the DNA, bringing the regulatory elements into close proximity. This spatial arrangement allows the enhancer-bound transcription factors to directly interact with the transcriptional machinery at the promoter, leading to enhanced transcriptional activity Enhancers can also possess cell type-specific or developmental stage-specific activity. This means that an enhancer may only be active in certain cell types or during specific stages of development, contributing to the precise regulation of gene expression. The specificity and activity of enhancers are governed by the combination of transcription factors that bind to them, creating a complex regulatory network that determines the timing, level, and specificity of gene expression. Additionally, enhancers can act synergistically with other enhancers or regulatory elements in a combinatorial manner. This cooperation between multiple enhancers allows for fine-tuning of gene expression patterns and enables cells to respond to a variety of environmental cues and signalling pathways. The combinatorial effects of enhancers provide a robust and dynamic mechanism for gene regulation, ensuring the proper functioning and adaptation of cells in different contexts, particularly when imparting tissue specificity in the form of phenotypic gene expression.

The term ‘silencer’ as used herein denotes a genetic regulatory element in a DNA sequence that reduces transcription from an associated promoter; typically they are the repressive counterparts of an enhancer. Silencers play a crucial role in reducing or repressing the transcriptional activity of an associated or adjacent promoter and contribute to the fine-tuning of gene expression. Silencers are typically located in proximity to the promoter region of the gene(s) they regulate. They can be found upstream (upstream silencers), downstream (downstream silencers), or even within introns of the gene. Like enhancers, silencers are not necessarily orientation-specific and can function regardless of their orientation relative to the gene. The main function of a silencer is to provide binding sites for transcription factors that have a repressive effect on gene transcription. When specific transcription factors recognize and bind to the silencer, they recruit co-repressor proteins or inhibit the binding of activator proteins to the promoter region. This interference leads to the repression of transcriptional activity from the associated promoter. Silencers can exert their repressive effects in multiple ways. They can directly interact with the transcriptional machinery at the promoter region, preventing the assembly of the necessary components for transcription initiation. Silencers can also induce chromatin modifications, such as the addition of methyl groups to DNA or the removal of acetyl groups from histones. These modifications alter the chromatin structure, making the DNA less accessible to the transcriptional machinery and inhibiting gene expression. Similar to enhancers, silencers can exhibit cell type-specific or developmental stage-specific activity. This means that silencers may only be active in certain cell types or during specific stages of development, adding another layer of complexity to gene regulation. The specific combination of transcription factors binding to the silencer determines its activity and repressive effect on gene transcription. Silencers can also function in a cooperative manner, interacting with other regulatory elements, such as other silencers or enhancers, to modulate gene expression. By working together, these elements fine-tune transcriptional activity and establish precise gene expression patterns in response to various signals and environmental cues. Hence, through the recruitment of repressive transcription factors and chromatin modifications, silencers function as dampeners of transcriptional activity, allowing cells to precisely regulate gene expression levels. Their cell type-specific and cooperative nature adds complexity to the gene regulatory network and ensures proper gene expression patterns during development and in response to different cellular contexts. In certain contexts a silencer may also be a bifunctional regulatory element that can also act as an enhancer, again depending upon cellular context.

The term ‘insulators’ is used to refer to genetic regulatory elements that have evolved as a complementary mechanism for structurally and functionally distinguishing regions of euchromatin from heterochromatin. Typically, insulator elements are positioned peripherally with respect to a given transcriptional unit—e.g. a gene. Insulators function by establishing boundaries between neighbouring transcriptional units to prevent encroachment by adjacent regions of heterochromatin. Insulators may also function as gatekeepers in permitting or preventing access to a transcription unit by transcriptional regulatory proteins. Insulators may serve at least two functions that contribute to cell-type specificity: (1) providing a protective shield against deleterious effects of neighbouring enhancer regions on the transcriptional activity of a gene, and (2) facilitating or to amplifying the activity of distantly positioned, multi-element enhancer complexes or locus control regions within a given transcriptional unit.

“Gene editing” refers to a type of genetic engineering in which the nucleotide sequence of a target polynucleotide is changed through introduction of deletions, insertions, or base substitutions to the polynucleotide sequence. CRISPR-Cas based gene editing is one way of achieving such changes to a target genomic sequence. Genome editing may include correcting or restoring a mutant gene. Genome editing may include knocking out a gene, such as a mutant gene or a normal gene. Genome editing may be used to treat disease or enhance tissue repair by changing the gene of interest. In some embodiments, the methods detailed herein are for use in somatic cells and not germ line cells.

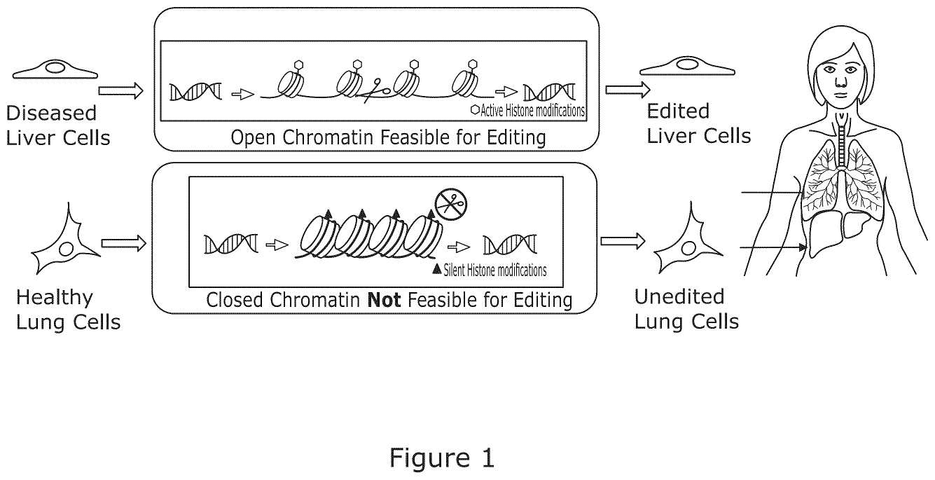

The ‘accessibility’ of a DNA comprising region of chromatin, also referred to as ‘accessible DNA’ interchangeably, refers to the ability of a particular locus within a chromosome of a cell to be contacted and modified by a particular DNA cleaving or modifying agent—such as an RNA-guided endonuclease complex. Without intending to limit the scope of the present invention, it is supposed that chromatin structure comprised within a given DNA region will affect the efficiency of genetic modification, such as through gene editing, for that particular DNA region. For example, the DNA region may be comprised within condensed heterochromatin that prevents or reduces access of the gene editing agent to the DNA in the region of interest. FIG. 1 schematically depicts the impact the chromatin structure is thought to have on the feasibility of editing. Accessibility can therefore be considered as a function of the quantity or efficiency of DNA cleavage or modification, such as via the action of a DNA endonuclease. Relative accessibility between two DNA regions can be determined by comparing (e.g., generating a ratio) of the amount of cleavage or modification between the two regions, or loci.

The term ‘chromatin’ refers to the condensation of genomic DNA into an organized complex of chromosomal DNA associated with histone proteins. found in eukaryotic cells. Heterochromatin refers to a condensed and tightly packed form of chromatin and is characterized by its transcriptionally repressive state, which prevents the expression of genes in these regions. It is typically located near the centromeres and telomeres of chromosomes, and plays important roles in chromosome organization, DNA replication, and overall genome stability. Heterochromatin can be distinguished from its less condensed counterpart, euchromatin, by its dark staining properties in microscopy and its relative inaccessibility to enzymes involved in DNA transcription and repair. Hence, as used herein the term ‘heterochromatin’ refers to transcriptionally inactive regions of a chromosomal DNA consisting of highly condensed DNA/Histone complexes, called nucleosomes, that are insensitive to endonuclease treatment, e.g. with DNAse I. Heterochromatin can be characterized by detecting the deacetylation states of Histone 3 and Histone 4 and the methylation state of Histone 3 at lysine 9 (i.e. H3K9 methylation). In contrast, ‘euchromatin’ refers to a more accessible genomic region enriched with less condensed chromatin. In some embodiments, a euchromatic region is a genomic region that is hypersensitive to nuclease digestion, e.g., by DNAse I or micrococcal nuclease. Thus, in some embodiments, euchromatic regions may be identified using DNase-Seq (DNase I hypersensitive sites sequencing), which is based on sequencing of regions sensitive to cleavage by DNase I. In some embodiments, a euchromatic region is a genomic region or locus that is relatively depleted of nucleosomes. Thus, in some embodiments, euchromatic regions may be identified using FAIRE-Seq (Formaldehyde-Assisted Isolation of Regulatory Elements), which is based on an observation that formaldehyde cross-linking is more efficient in nucleosome-bound DNA than it is in nucleosome-depleted regions of the genome. This method segregates the non-cross-linked DNA that is usually found in open chromatin, which is then sequenced. The protocol typically involves cross linking, phenol extraction and sequencing DNA in aqueous phase.

In some embodiments of the invention, a euchromatic region is comprised of a genomic region that is enriched in methylated histones (e.g., methylated Histone H1, H2A, H2B, H3 or H4) compared to an appropriate control. In some embodiments, an appropriate control is a corresponding genomic region in a reference cell type or tissue, e.g. an undifferentiated or less differentiated cell, or terminally differentiated cell.

The terms ‘guide molecule’ and ‘guide RNA’ or ‘gRNA’ are used interchangeably herein to refer to RNA-based molecules that are capable of forming a complex with an RNA-guided endonuclease complex, such as a CRISPR-Cas protein. A gRNA typically comprises a guide sequence having sufficient complementarity with a target nucleic acid sequence to hybridize with the target nucleic acid sequence and direct sequence-specific binding of the complex to the target nucleic acid sequence. Typical gRNA molecules include a targeting sequence, which binds to the complementary DNA sequence, and a Cas protein binding scaffold region, which interacts with the Cas enzyme (or equivalent or derivative thereof). The guide molecule or guide RNA may encompass RNA-based molecules having one or more chemical modifications, including synthetic bases, or by chemical linking two ribonucleotides or by replacement of one or more ribonucleotides with one or more deoxyribonucleotides). For example, chemical modifications such as 2′-O-methyl or phosphorothioate modifications can be introduced to increase gRNA stability. The disclosure provides a guide nucleic acid suitable for use in a CRISPR/Cas system. A gRNA binds to a Cas protein via the scaffold region and targets the Cas protein to a specific location within a target nucleic acid. In some cases, a guide nucleic acid comprises a single nucleic acid molecule, referred to as a single guide nucleic acid (sgRNA). Alternatively, a guide nucleic acid comprises two separate nucleic acid molecules, referred to as a double guide nucleic acid.

The synthesis of gRNA typically involves two main steps: in vitro transcription and purification. In the in vitro transcription step, a DNA template containing the scaffold region, targeting sequence, and a promoter recognized by an RNA polymerase is used. This template is subjected to transcription using an RNA polymerase, resulting in the synthesis of a single-stranded RNA molecule, which is the gRNA. After the in vitro transcription, the gRNA is usually purified to remove impurities and any remaining DNA template or RNA polymerase. Common purification methods include column purification, precipitation, or enzymatic treatment to eliminate contaminants. The purified gRNA is then typically quantified and quality checked using spectrophotometry or gel electrophoresis. Modified versions of Cas enzymes, such as Cas9 variants or other CRISPR systems (e.g., Cas12a, MAD-7, Cas13), have been developed, which may require specific modifications or considerations during gRNA synthesis. It will be appreciated that gRNA synthesis protocols are known to the skilled person (for example see Doensch et al. Nat Biotechnol. (2014) December; 32(12): 1262-1267).

In certain embodiments, the guide molecule comprises (1) a guide sequence capable of hybridizing to a target locus that has cell, tissue or phenotype specificity, and (2) a tracr mate or direct repeat sequence whereby the direct repeat sequence is located upstream (i.e., 5′) or downstream (i.e. 3′) from the guide sequence. In a specific embodiment the portion of the sequence that is essential or critical for recognition and/or hybridization to the sequence at the target locus (the “seed sequence”) of the guide sequence is approximately within the first 10 nucleotides of the guide sequence.

According to the present invention, homology to any of the nucleic acid sequences, such as the gRNA sequences described herein, is not limited simply to 100%, 99%, 98%, 97%, 95%, 90%, 85% or even 80% sequence identity. Optimal alignments may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). Many nucleic acid sequences can demonstrate biochemical equivalence to each other despite having apparently low sequence identity. In the present invention homologous nucleic acid sequences are considered to be those that will hybridise to common target sequence under conditions of low stringency (Sambrook J. et al, Molecular Cloning: a Laboratory Manual, Cold Spring Harbor Press, Cold Spring Harbor, NY). However, it may be desired in some cases to distinguish between two sequences which can hybridise to common target sequence but contain some mismatches—an “inexact match”, “imperfect match”, or “inexact complementarity”—and two sequences which can hybridise to the target with no mismatches—an “exact match”, “perfect match”, or “exact complementarity”. Further, possible degrees of mismatch are considered. A sequence capable of hybridizing with a given target sequence is referred to as the “complement” of the given sequence. In specific embodiments, When comparing DNA and RNA, thymine (T) and uracil (U) may be considered equivalent

The term “target sequence”, in the context of formation of an RNA-guided endonuclease complex, refers to a sequence to which a guide sequence is configured to target, e.g. have complementarity with where hybridization between a target sequence and a guide sequence promotes the formation of a endonuclease complex, such as a CRISPR complex. As mentioned above, the portion of the guide sequence that hybridises to the target sequence may be termed a “seed sequence”. A target sequence may comprise any polynucleotide, such as DNA or RNA polynucleotides. In specific embodiments, a target sequence is located in the nucleus or cytoplasm of a cell, and may include nucleic acids in or from mitochondrial, organelles, vesicles, liposomes or particles present within the cell. Typically the target sequence will be comprised within a tissue specific region of a chromosome within a cell. Suitably, the target sequence will be comprised within an accessible chromatin region, such as within a locus that is active within a specific cell type, and that is uniquely accessible within the cell-type or tissue type, thereby conferring a level of phenotypic specificity to a gRNA that binds to the target sequence.

In embodiments of the invention the target sequence may be comprised within candidate nucleic acid sequences and/or tissue specific candidate sequences identified via the methods of the present invention.

Various RNA guided endonucleases are consistent with the methods of the present disclosure. Typically these sequence guided endonucleases fall within the general disclosure of a CRISPR/Cas endonuclease system. In general, the “CRISPR/Cas endonuclease system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as those described in more detail below. In general, a CRISPR/Cas endonuclease system is characterized by elements that promote the formation of a CRISPR complex at the site of a target sequence as defined herein.

In particular embodiments of the invention, the target sequence may be associated with a PAM (protospacer adjacent motif); that is, a short sequence recognized by the CRISPR complex as the site for cleavage of the DNA. The precise sequence and length requirements for the PAM differ depending on the CRISPR enzyme used, but PAMs are typically 2-5 base pair sequences located adjacent to a protospacer—i.e. the target sequence.

In some embodiments of the invention, the endonuclease is selected from Cas9, Cpfl, c2cl, C2c2, Casl3, c2c3, Casl, CaslB, Cas2, Cas3, Cas4, Cas5, Cas5e (CasD), Cas6, Cas6e, Cas6f, Cas7, Cas8, Cas8a, Cas8al, Cas8a2, Cas8b, Cas8c, Csnl, Csxl2, Cas9, Cas10, Cas10d, Cas12a, Cas12b, Cas12c, Cas12d, Cas12e, Cas13a, Cas13b, Cas13c, Cas13d, CasF, CasG, CasH, Csyl, Csy2, Csy3, Csel (CasA), Cse2 (CasB), Cse3 (CasE), Cse4 (CasC), Cse5, Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csx17, Csx14, CsxIO, Csxl6, CsaX, Csx3, Csxl, Csx15, Csfl, Csf2, Csf3, Csf4, Cul966, or a derivative thereof, a variant thereof, and a fragment thereof, wherein a fragment of the RNA-guided endonuclease is a protein recognizable by a person of skill in the art as retaining some or all of the common activity or having sufficient sequence identity as a protein listed above. Alternatively, some RNA-guided endonucleases are modified versions of the wildtype form, for example, comprising an amino acid change such as a deletion, insertion, substitution, variant, mutation, fusion, chimera, or any combination thereof, relative to a wild-type version of the protein. In some embodiments, the endonuclease comprises a region exhibiting at least 70% identity over at least 70% of its residues to a Cas9 domain or a Cpfl domain. In particular embodiments, the Cas9 is selected from the group consisting of SpCas9 SaCas9, StCas9, NmCas9, FnCas9, and CjCas9. In other embodiments, the region is a Cpfl domain, or a derivative thereof including MAD-7.

RNA-guided nucleases of the types disclosed herein are derived either directly or modified from a number of possible sources. Such endonucleases may be eubacterial, archaeal, or thermostable in origin. In specific embodiments, the programmable endonuclease is derived from a species selected from the group consisting of Streptococcus pyogenes (S. pyogenes), Streptococcus thermophilus, Streptococcus sp., Staphylococcus aureus, Nocardiopsis dassonvillei, Streptomyces pristinae spiralis, Streptomyces viridochromo genes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Pseudomonas aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Leptotrichia shahii, Prevotella, and Francisella novicida.

‘Subject’, ‘individual subject’ or ‘patient’ as used herein, may mean either a human or non-human animal. The term includes, but is not limited to, mammals (e.g., humans, other primates, pigs, rodents (e.g., mice and rats or hamsters), rabbits, guinea pigs, cows, horses, cats, dogs, sheep, and goats). In an embodiment, the subject is a human. In other embodiments, the subject is agricultural livestock or poultry. In another embodiment, the subject is a fish, including farmed fish stocks.

As used herein, the term “on-target” editing event refers to a gene edit that occurs at a location of a target gene, sequence or nucleic acid to which a target specific gene editor complementarily binds, whereas, the term “off-target” as used herein refers to a sequence or position of a target or non-target gene or nucleic acid to which a target-specific gene editor fully or partially binds, but where undesired editing activity occurs. Consequently, off-target effects are defined as undesired editing outcomes, outside of their intended target scope, i.e., unintentional cleavage and/or mutations at non-directed genomic therapeutic sites. The non-directed genomic site often has a similar, but not an identical, sequence to the directed target genomic site. Hence, the non-directed genomic site is also known as an off-target site, even though the target sequence may be similar. In CRISPR-Cas editing, off-target sites may be identified for example by determining the number of base mismatches between the guide RNA and the off-target site. High mismatches refer to a mismatch of two or more, for example three, four, five, six, etc. nucleotides. In contrast, target sites typically have no mismatch or a very low mismatch, for example a maximum of two mismatches, suitably only a single mismatch. Hence, in a specific embodiment, an off-target editing event corresponds to a gene editing occurring at a sequence or location of a gene or nucleic acid that is not targeted by a target specific base editor, or a nucleic acid sequence that has less than 100% sequence homology with the nucleic acid sequence of the on-target. Suitably, the off target site has sequence homology with the target site of less than 99%, less than 98%, less than 95%, less than 90%, less than 85%, or even less than 80%. The off-target nucleic acid sequence having less than 100% sequence homology with the on-target nucleic acid sequence is typically a nucleic acid sequence similar to the on-target nucleic acid sequence but may include one or more additional nucleotides and/or has one or more nucleotides deleted.

In accordance with an embodiment of the present invention, a method for making a novel gRNA for use with an RNA-guided endonuclease. The gRNA comprises a nucleic acid sequence that is configured to hybridise with a target nucleic acid sequence within the genome of a cell, wherein the target nucleic acid sequence is characterised as being comprised within a locus that is active within a specific cell type. Novel gRNAs that impart tissue or cell-type specificity may be used in therapeutic gene editing to treat a range of diseases in patients, or to effect other changes such as desirable traits in non-human subjects.

The invention provides a method comprising the steps of:
i. identifying one or more candidate nucleic acid sequences that are unique to a specific cell type as compared to a control cell that is selected from a different cell type, thereby defining tissue specific candidate nucleic acid sequences;
ii. identifying a subset of the candidate nucleic acid sequences of (i) that are comprised within an accessible chromatin region of the genome within the specific cell type; and
iii. synthesising a gRNA that hybridises to one or more of the candidate nucleic acid sequences identified in (ii).

Identification of accessible chromatin regions comprised within loci of the genome of a given specific cell-type or tissue involves identification within the cell or tissue of a sequences that are highly specific to that cell type or tissue. These target sequences represent promising candidates for the design of complementary gRNAs that will hybridise to them and thereby direct RNA-guided endonuclease activity to these cell/tissue specific loci. Hence, such target sequences are referred to as hyper targets. In one embodiment, the methods of the present invention utilize an in silico approach to screen for tissue specific hyper targets in the cell type of choice for a given gene of interest. The algorithm may be configured to assess data inputs that are derived from one of more of the following sources:

Identification of unique regulatory elements including but not limited to IncRNAs, mi-RNAs, enhancers, repressors, transcription factors, transcription factor binding sites, RNA binding proteins etc. that impact the gene of interest.
Screening across all validated databases such as ENCODE, Vista, Slidebase, CAGE (DB), NCIB, Nature, Protein Atlas, Gene cards, HACER, UCSC genome browser, etc.
Comparative analysis of the epigenetic profiles—including chromatin structure and methylation status-across cell types of the chosen unique region of interest and in comparison to non-target cell lines.

In one embodiment of the invention, epigenetic profile data (standardized and normalized) may be downloaded from the Encyclopedia of DNA Elements or ‘Encode’ database as per the desired cell-type in Homo sapiens (Nature (2012) September 6; 489(7414):57-74). The analysis identifies a plurality of candidate hyper target sequences within the desired cell lines. A normalization function may be carried out using max and min values for the target chromosome, if max is M and min is N for a given chromosome, and if the value of epigenetics at location is X a Normalization Value=(X−N)/(M−N).

According to embodiments of the invention, epigenetic features considered for the hyper target identification and validation, as well as for profiling the on-target and off-target may include any one or more of the following indicators of accessible chromatin:

RNA sequencing data: RNA sequencing (RNA-seq) data can be utilized to assess chromatin accessibility, suitably through a technique called RNA-seq-based Assay for Transposase-Accessible Chromatin using sequencing (RNA-Seq based ATAC Seq). This approach combines the principles of RNA-seq and ATAC-seq (Assay for Transposase-Accessible Chromatin) to gain insights into chromatin accessibility from RNA-seq data. The basic principles of RNA-seq based ATAC seq include the preparation of an RNA-seq library by isolating RNA from a sample, converting it into cDNA, and generating sequencing libraries using standard RNA-seq protocols. The resulting libraries contain information about the RNA expression levels in the sample. Low abundance transcripts in the libraries are indicative of potential regulatory regions that may be cell-type or tissue type specific. Hence, low abundance transcripts are typically selected. These transcripts may correspond to non-coding RNAs or unannotated regions of the genome and, in turn, such regions are likely to be associated with chromatin accessibility changes. The selected low-abundance transcripts may be reverse transcribed into DNA and then subjected to an ATAC-seq-like protocol. This involves fragmenting the DNA using a transposase enzyme and adding sequencing adapters. The transposase preferentially cleaves accessible regions of the chromatin, allowing the sequencing adapters to be added to the sites of chromatin accessibility. These resulting ATAC-seq libraries, which now contain information about chromatin accessibility, can be sequenced using high-throughput sequencing platforms. The sequencing data is then analyzed using bioinformatics tools specifically developed for ATAC-seq analysis. This allows the identification of regions of open chromatin in the genome. The final step involves integrating the RNA-seq data and the ATAC-seq data. By correlating the expression levels of the selected low-abundance transcripts with the accessibility of corresponding genomic regions, it is possible to gain insights into the relationship between gene expression and chromatin accessibility. This integration can provide valuable information about the regulatory elements and potential transcription factor binding sites that influence gene expression and that can be determinative of tissue or cell type specificity.

CCCTC-binding factor (CTCF) data: CTCF is a highly conserved zinc finger protein and transcription factor. It can function as a transcriptional activator, a repressor or an insulator protein, blocking the communication between enhancers and promoters. CTCF can also recruit other transcription factors while bound to chromatin domain boundaries. CTCF plays a crucial role in chromatin architecture, and its presence can define chromatin accessibility. This parameter may be used to assess the chromatin accessibility of the tissue/cell specific region within the tissue/cell of interest and in potential off target tissues/cells. CTCF data can be utilized to determine chromatin accessibility in combination with other techniques as follows:
1. ChIP-seq: Chromatin immunoprecipitation sequencing (ChIP-seq) is a widely used technique to identify DNA regions bound by specific proteins, such as CTCF. By performing ChIP-seq experiments with CTCF antibodies, it is possible to map the genomic locations where CTCF binds. These CTCF binding sites can provide insights into the organization and accessibility of chromatin. CTCF binding at specific loci can indicate the presence of chromatin loops or boundaries that influence the accessibility of neighbouring regions.
2. Integration with ATAC-seq: ATAC-seq (Assay for Transposase-Accessible Chromatin using sequencing) can be used to directly assess chromatin accessibility by mapping open chromatin regions. Integration of CTCF ChIP-seq data with ATAC-seq data can reveal the relationship between CTCF binding and chromatin accessibility. Regions of open chromatin that overlap with CTCF binding sites can suggest that CTCF may contribute to maintaining or influencing chromatin accessibility in those regions.
3. Motif analysis: CTCF has a well-characterized DNA-binding motif, which consists of a specific sequence pattern. By analyzing genomic regions for the presence of the CTCF motif, researchers can identify potential CTCF binding sites. These predicted binding sites can serve as indicators of accessible chromatin regions, as CTCF tends to preferentially bind to regions with open chromatin.
4. Hi-C and 3C-based techniques: Hi-C and other 3C-based (chromosome conformation capture) techniques provide insights into the three-dimensional organization of the genome. CTCF is known to participate in the formation of chromatin loops and interactions between distant genomic regions. By integrating CTCF ChIP-seq data with Hi-C or 3C-based data, researchers can identify CTCF-mediated chromatin interactions, which can help infer chromatin accessibility. CTCF binding sites often mark the boundaries of chromatin loops, and the interactions facilitated by CTCF can influence the accessibility of the regions within the loops.

A deoxyribonuclease (DNase, for short) is an enzyme that catalyzes the hydrolytic cleavage of phosphodiester linkages in the DNA backbone, thus degrading DNA. Deoxyribonucleases are one type of nuclease, a generic term for enzymes capable of hydrolyzing phosphodiester bonds that link nucleotides. DNase activity is one way to assess chromatin accessibility and to define the importance of the tissue/cell specific region within the tissue/cell of interest and in potential off target tissues/cell. DNase accessibility data can be used to determine chromatin accessibility by:
1. DNase-seq or DNase hypersensitivity assay: In a DNase-seq experiment, cells or tissues are treated with DNase I to selectively cleave open chromatin regions. After DNase treatment, DNA fragments from accessible regions are isolated, sequenced, and mapped to the reference genome. This generates a DNase-seq dataset that represents the regions of open chromatin.
2. Peak calling: The DNase-seq data is analyzed using bioinformatics tools to identify regions of increased DNase cleavage, also known as DNase hypersensitive sites (DHSs) or peaks. These DHSs correspond to accessible chromatin regions, as they are more susceptible to DNase cleavage compared to closed or compacted chromatin regions.
3. Integration with other genomic data: DNase accessibility data may be integrated with other genomic datasets, such as transcription factor binding data, histone modification data, or gene expression data. By overlapping DNase hypersensitive sites with these datasets, it is possible to can gain insights into the functional significance of the accessible regions. For example, co-localization of DNase hypersensitive sites with transcription factor binding motifs can suggest potential regulatory elements involved in gene regulation or tissue or cell type specificity.
4. Regulatory element identification: DNase accessibility data can help identify regulatory elements, including promoters, enhancers, and other cis-regulatory elements. Promoters are typically characterized by open chromatin regions around the transcription start site, which can be detected using DNase accessibility data. Enhancers, which are distal regulatory elements, often display DNase hypersensitivity and can be located by examining accessible regions far from annotated promoters.
5. Comparative analysis: DNase accessibility data can be compared across different cell types, tissues, or conditions to identify cell-specific or context-specific chromatin accessibility patterns. By comparing DNase-seq profiles, it is possible to infer dynamic changes in chromatin accessibility associated with cellular processes, development, or responses to external stimuli.
6. Functional analysis: Once regions of open chromatin are identified, functional characterization can be performed to understand their roles in gene regulation. This may involve assessing transcription factor binding, analysing gene expression changes upon perturbation of the accessible regions, or investigating the impact of genetic variants within the accessible regions on gene expression or phenotype through in vivo expression characterisation assays.

Histone modification data: To assess chromatin accessibility and to define the importance of the tissue/cell specific region within the tissue/cell of interest and in potential off target tissues/cells methylation and/or acetylation of histones within the region of interest can provide information regarding (a) Promoter accessibility—H3K4me3, H3K9me3; (b) Gene bodies—H3K36me3, H3K27me3, and (c) gene regulatory elements-H3K27Ac, H3K4me1. Suitable techniques include chromatin immunoprecipitation followed by sequencing (ChIP-seq).

As previously mentioned, chromosome conformation capture (3C) methodologies such as Hi-C analysis may be used to assess chromatin accessibility, 3D organization of the genome and interconnectivity to assess the regulation/connection of the tissue/cell specific region within the target gene of interest and/other associated genes within the tissue/cell of interest and in potential off target tissues/cells (Lieberman-Aiden et al. Science. 2009 Oct. 9; 326(5950): 289-293). ATAC-seq (Assay for Transposase-Accessible Chromatin with high-throughput sequencing) approaches may also be used alone or in combination with other methodologies to investigate chromatin accessibility in a sample.

Epigenetic analysis based upon data obtained via any one or more of the assays described herein may be carried out using bioinformatics approaches known to the skilled person, such as using tools such as pyBigWig, a Python extension written in C programming language, which allows for quick access to bigBed files and access to and creation of bigWig files. The pyBigWig Python package is a powerful library that provides a Python interface to handle bigWig files. BigWig files are a binary format commonly used in genomics and bioinformatics to store large genomic data, such as genome-wide signal data or coverage tracks. Hence, the pyBigWig package allows reading, writing, and manipulation of bigWig files within Python code. It provides an easy-to-use interface to access the data stored in the files, as well as perform various operations on the genomic data. One of the primary advantages of using pyBigWig is its efficiency in working with large genomic datasets. It leverages the libBigWig C library, which is a fast and memory-efficient implementation for reading and writing bigWig files. By utilizing this library, pyBigWig provides efficient I/O operations and enables high-performance processing of genomic data, such as for reading signal data values for a specified genomic region. In one embodiment, a pyBigWig Python package in bioconda (https://bioconda.github.io/) is used to analyze the epigenetics for the region of interest to assess tissue/cell specificity. Selection of the unique hyper target candidate sequences/tissue specific signatures aids in the design of tissue specific gRNAs that can target and bind to the given targets.

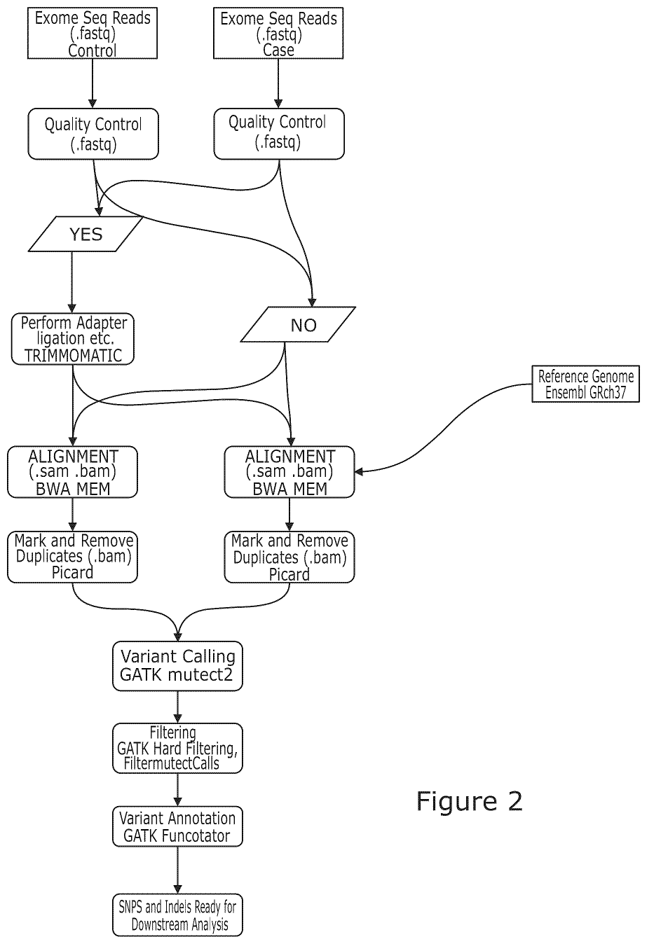

An exemplary whole exome sequence (WES) bioinformatics analysis pipeline is shown in FIG. 2. After receiving a whole exome sequence reads file (.fastq format) the sequenced data files generated from unedited (control) and edited cells are identified and segregated from each other. The files will typically undergo a quality check to evaluate per sequence and per base quality scores along with sequence length distribution and adapter content. The next step is to align the reads to a reference genome assembly, for example the Genome Reference Consortium Human Build 37 (GRCh37). This step creates data processed in Sequence Alignment/Map format (.sam) and the corresponding compressed binary version (.bam) files. After aligning these files to the reference genome any duplicate reads are removed for the .bam file to get the distinct reads using PICARD tools (https://broadinstitute.github.io/picard/). All the above steps may be performed in parallel on both the control and edited cell data files. The.bam files after removing duplicates are then used as an input together into a Bayesian somatic genotyping model to identify somatic short mutations via local assembly of haplotypes, e.g. via use of a tool such as the GATK mutect2 tool for variant calling (available from https://gatk.broadinstitute.org/). While finding these variants multiple steps may be used to hard filter the variants that are not caused due to the editing apparatus or the delivery mechanism. The filtered data is then annotated using a functional annotator that analyzes given variants for their function (as retrieved from a set of data sources—e.g. literature) and produces the analysis in a specified output file—for example, Funcotator (available from https://gatk.broadinstitute.org/). This analysis provides an output that includes all the short mutations, such as SNPs and Indels, that have been created in the given cell line because of the gene editing event. The data may be represented in graphical form to the user or in any other suitable format that provides information regarding the efficiency and efficacy of the edit, including whether off-target events have occurred.

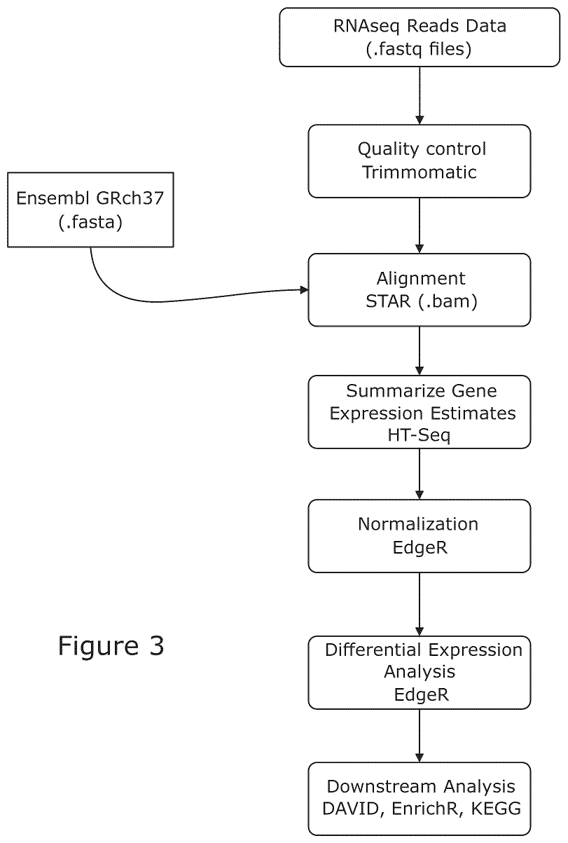

An exemplary RNAseq bioinformatics analysis pipeline is shown in FIG. 3. RNAseq Read data files which are received in .fastq format post sequencing from unedited (control) cells and edited cells are analyzed through FastQC to check the quality of the analysis. The files will typically be evaluated for per sequence and per base quality scores along with sequence length distribution and adapter content. The next step is to align the reads using for example the STAR (Spliced Transcripts Alignment to a Reference) Aligner to a reference genome assembly, for example the Genome Reference Consortium Human Build 37 (GRCh37). This step creates data processed in Sequence Alignment/Map format (.sam) and the corresponding compressed binary version (.bam) files. Gene expression is then summarised using HT-Seq, followed by normalisation of expression values using EdgeR. These normalised expression value files are then analyzed for differential expression analysis using same EdgeR tool. The output of all of previous steps is then used to analyze the high-level functions and utilities of the cell, the organism and the ecosystem, from genomic to molecular-level using gene set enrichment tools such as Enrichr (available from https://maayanlab.cloud/Enrichr/) and processes such as KEGG mapping (available from https://www.genome.jp/kegg/).

Once a suitable hyper target has been identified, screening of canonical (NGG) and other potential PAMs suitable for the respective gene editing enzyme of interest (e.g. Cas or a derivative thereof) occurs across the hyper target location.

In embodiments of the invention a gRNA that hybridises to a tissue/cell-type specific target nucleic acid sequence, e.g. within the hyper target, is synthesised. Suitably the gRNA is an sgRNA. Further steps may be used to validate the target specificity that include one or more in vitro and in vivo assays.

An exemplary methodology for selection of liver specific editable targets is depicted in FIG. 2 and FIG. 3. Liver specific editable targets are screened by the Hele GUIDE Platform. Once the target has been identified, for example one impacting the expression of the DD4 (AKR1C4) gene, liver specific gene editing apparatus is designed (for example select gRNA combined with Cas9 ribonucleoprotein (RNP)). Analyses of target and non-target cell lines is carried out by for example Sanger sequencing and T7E1 assay. The cells are then subjected to monoclonal expansion to perform edit analysis. Typically, the target cells show high edit percentage compared to non-target cells which show no or very low edit percentages.

Hence, according to embodiments of the invention methods for the production of gRNAs that have tissue type, cell type or other phenotype target specificity are provided. In these methods the gRNA is able to hybridise with a tissue type, cell type or other phenotype specific target within the genome of a cell and facilitate a gene editing event within the cell catalysed by a RNA-guided endonuclease, such as a Cas protein or derivative thereof. The gRNA comprises a sequence that is complementary to and hybridises with the tissue type, cell type or other phenotype specific target sequence identified following an analysis of the target cell to prioritise targets sequences that are within regions that have epigenetic specificity to the desired the tissue type, cell type or other phenotype.

In a specific embodiment of the invention, there is provided a method of modifying nucleic acid sequences associated with or at a target locus of interest wherein the target is a cell-type, tissue type or phenotype specific locus. The method comprises delivering to said nucleic acid or locus a non-naturally occurring or engineered composition comprising a RNA-guided endonuclease (such as a CRISPR-Cas effector protein or a derivative thereof) and one or more associated nucleic acid components (such as a gRNA or an sgRNA), and wherein the CRISPR-Cas effector protein—nucleic acid form a complex that is capable of modification of sequences associated with or at the cell-type, tissue type or phenotype target locus of interest. In one embodiment, the modification comprises the introduction of a strand break. In another embodiment, the modification comprises a base substitution. In another embodiment, the modification comprises modulating gene expression, including but not limited to, increasing or decreasing expression. In another embodiment, the modification comprises a change in methylation. In certain embodiments, the target nucleic acid comprises DNA. In certain embodiments, the target nucleic acid comprises RNA. In certain embodiments, a non-target nucleic acid is collaterally modified. In certain embodiments, the target nucleic acid is in a prokaryotic cell. In other embodiments, the target nucleic acid is in a eukaryotic cell, suitably a plant or animal cell, most suitably a human cell.

In some embodiments, the polynucleotide encoding one or more features of the RNA-guided endonuclease system and/or nucleic acid components thereof, such as guide sequences, can be expressed from a vector in vivo or in vitro or from a suitable polynucleotide in a cell-free in vitro system. Vectors can be designed for expression of one or more elements of the RNA-guided endonuclease system and/or nucleic acid components thereof as described herein (e.g. nucleic acid transcripts, proteins, enzymes, and combinations thereof) in a suitable host cell. The suitable host cell may be a prokaryotic or eukaryotic cell, including but not limited to, bacterial cells, yeast cells, insect cells, and mammalian cells. The vectors can be viral-based or non-viral based. Suitable bacterial cells include but are not limited to bacterial cells from the bacteria of the species Escherichia coli. Many suitable strains of E. coli are known in the art for expression of vectors. These include, but are not limited to Pir1, Stbl2, Stbl3, Stbl4, TOP10, XL1 Blue, and XL10 Gold. In contrast, in vitro translation of the RNA-guided endonuclease can be stand-alone (e.g. translation of a purified polyribonucleotide) or linked/coupled to transcription. In some aspects, the cell-free (or in vitro) translation system can include extracts from rabbit reticulocytes, wheat germ, and/or E. coli.

Also described herein are pharmaceutical formulations that can contain an amount, effective amount, and/or least effective amount, and/or therapeutically effective amount of one or more RNA-guided endonuclease system and/or nucleic acid components thereof, compounds, molecules, compositions, vectors, vector systems, cells, or a combination thereof (which are also referred to as the primary active agent or ingredient elsewhere herein) described in greater detail elsewhere herein a pharmaceutically acceptable carrier. In some embodiments, the pharmaceutical formulation can include, such as an active ingredient, an RNA-guided endonuclease system and/or nucleic acid components thereof, for example as part of a CRISPR-Cas system, a vector or vector system containing the system and/or component(s) thereof, a cell modified by the system and/or component(s) thereof, a cell containing the system and/or component(s) thereof, a cell capable of producing particles containing the system and/or component(s) thereof, particles and other delivery compositions containing or otherwise incorporating or associating with the RNA-guided endonuclease system and/or component(s) thereof, and combinations thereof. The pharmaceutical formulations described herein can be administered via any suitable method or route to a subject in need thereof as would be understood by a skilled person.

The invention is further illustrated by the following non-limiting examples.

### EXAMPLES

### Example 1

Identification of a Unique Liver Hepatocyte Specific Sequences for Use in gRNA Synthesis to Target the Liver Gene AKR1C4 (DD4)

The AKR1C aldo-keto reductases (AKR1C1-AKR1C4) are enzymes that interconvert steroidal hormones between their active and inactive forms. They can regulate the occupancy and trans-activation of the androgen, estrogen and progesterone receptors. The various AKR1C isoforms also have important roles in the production and inactivation of neurosteroids and prostaglandins, and in the metabolism of xenobiotics. Hence, they represent important emerging drug targets for the development of agents for the treatment of hormone-dependent forms of cancer, like breast, prostate and endometrial cancers, and other diseases, like premenstrual syndrome, endometriosis, catamenial epilepsy and depressive disorders.

The present objective is to exploit unique regulatory elements to design tissue-specific gRNAs for CRISPR-Cas-based gene modulation of the AKR1C4 (DD4) gene. A unique Transcription Factor Binding Site (TFBS) was selected as a candidate hyper target site for tissue specificity in liver impacting expression of the AKR1C4 gene. The binding site of transcription factor HNF-4 (−701 to −684 nucleotides) is unique to AKR1C4 in chromosome 10 and is only repeated 3 times across the whole human genome.

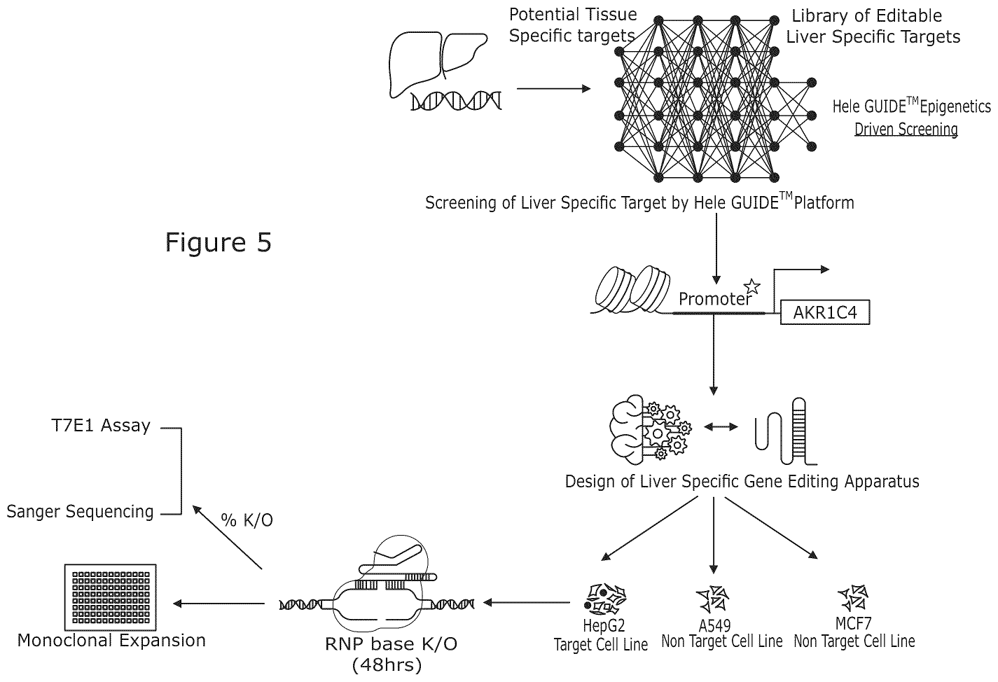

The unique TFBS was identified using the following assumptions derived from bioinformatic analysis of the DD4 (AKR1C4) gene. The general experimental approach is summarised in FIG. 5.
Identification of a unique binding motif of HNF1, HNF4 transcription factors in the promoter of DD4 gene (AKR1C4) within hepatocytes.
The HNF-4 motif is present on Chromosome 10:5236724-5236741 (−684 to −701) above the transcription start site (TSS) of DD4.
HNF-4 was identified as a unique site, present in only three known locations across the human genome.
HNF-1 motif is present on Chromosome 10:5236743-5236759 (−666 to −682) above the TSS of DD4.
HNF-4 unique to liver; RNA expression highest in liver: 100%, HNF-4 RNA expression in liver: 118 nTPM; HepG2: 114.8 nTPM.
HNF-1 RNA expression is highest in liver: 35nTPM; HNF-1 RNA expression in HepG2: 32.5 nTPM.
DD4 RNA expression in liver (723.4 nTPM), colon (0 nTPM), lung (0 nTPM); highly enriched in hepatocytes.
Cell line based RNA expression of DD4—HDLM2 (lymphoid: 6.6 nTPM), HepG2 (Hepatocytes: 1.1 nTPM), Caco2 (colon: 0 nTPM), A549 (lung: 0.8 nTPM).
DD4 does not show much expression in immortalised liver cells.

Epigenetics metrics used to analyse the upstream DD4 region and included the following:
Liver: Promoter region including both motifs (35bp) used H3K27Ac, H3K4Me1, H3K4Me3 and H3K36Me3 which should be higher in comparison to lung/colon reference tissue
Liver: Promoter region including both motifs (35bp) H3K9Me3, H3K27Me3 should be lower in liver in comparison to reference colon/lung tissue

Table 1 shows the results of epigenetic analysis of DD4 hyper targeted region in human hepatocytes (HepG2 cells) compared to reference lung tissue (a549 cells).

TABLE 1

| | Liver | Lung |
|---|---|---|
| Metric | (HepG2) | (a549) |
| H3K27Ac | low | high |
| H3K4Me1 | high | low |
| H3K4Me3 | low | high |
| H3K36Me3 | low | high |
| H3K9Me3 | low | high |
| H3K27Me3 | low | high |
| DNAse1 | high | low |

Based upon this analysis two putative sequences were identified for inclusion in gRNAs in order to confer liver specific targeting of the DD4 gene (AKR1C4) as follows:

SEQ ID NO: 1
5′-GAUUAAAUAGGCACAUGUGU-3′
SEQ ID NO: 2
5′-AAAAUUAUUUUAGAUUAAAU-3′

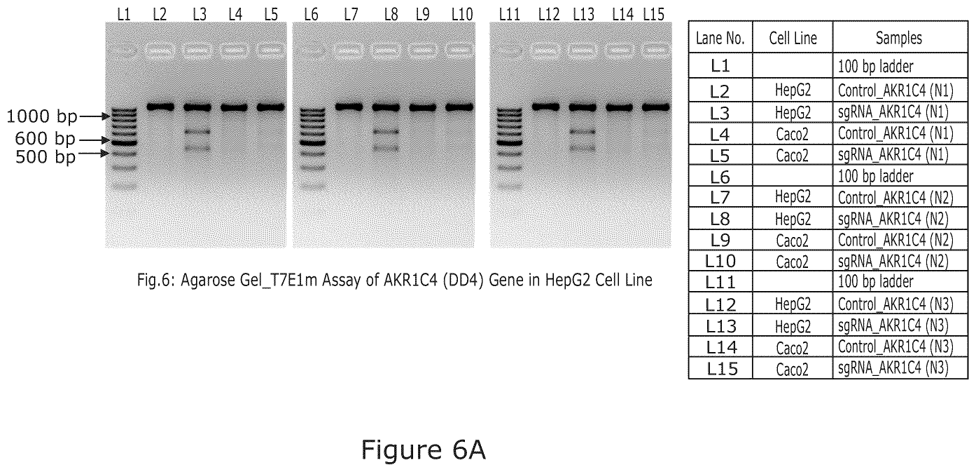

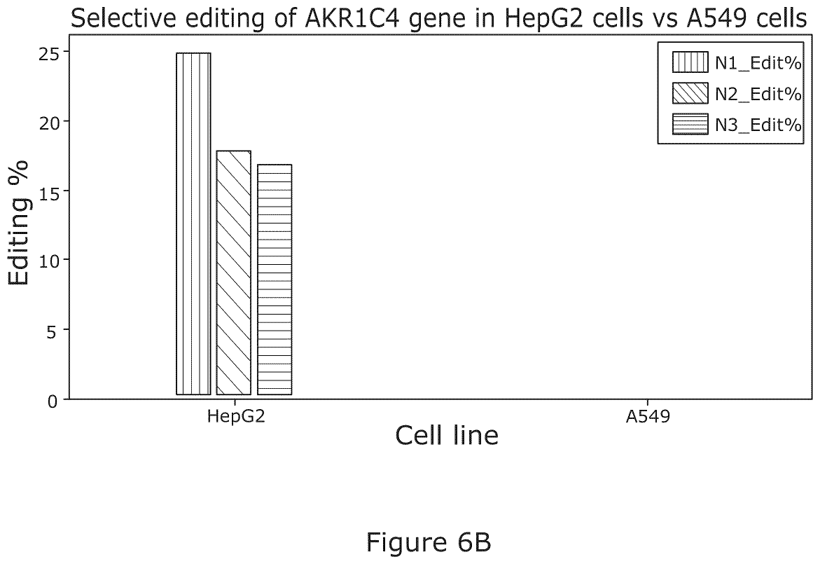

FIG. 6A shows the results of an in vitro editing assay using SEQ ID NO. 1 in target liver cell (HepG2) and non-target lung (A549) and breast cancer (MCF7) cells. Target liver cells show higher editing than non-target cells over three runs (N1 to N3). The graph in FIG. 6B shows the edit percentage in liver cells is 82.8% higher than lung cells (A549) and 77.4% higher than breast cancer cells (MCF7).

Further highly selective editing was shown in a T7E1 assay in a comparison between HepG2 (representative of liver cells) as the target cell line to display tissue specificity, and A549 lung non-target cells (see FIG. 6A and B). The results support the determination of the gRNAs as highly selective to the DD4 gene in liver.

### Example 2

Identification of a Unique Liver Hepatocyte Specific Sequences for Use in gRNA Synthesis to Target the Liver Gene TTR

Transthyretin (TTR) is a tetrameric protein synthesized predominantly in the liver and then secreted into the plasma. TTR molecules can misfold and form amyloid fibrils in the heart and peripheral nerves, either as a result of gene variants in TTR or as an ageing-related phenomenon, which can lead to amyloid TTR (ATTR) amyloidosis. Some of the proposed strategies to treat ATTR amyloidosis include blocking TTR synthesis in the liver, stabilizing TTR tetramers or disrupting TTR fibrils. TTR silencing has been proposed as a viable treatment for ATTR amyloidosis which makes the TTR gene a candidate for genome editing with CRISPR-Cas to reduce TTR gene expression.

The TTR gene is transcriptionally regulated by two DNA regions: a proximal −150 to −90 bp promoter region and a distal 100-nucleotide enhancer located −-2 kb upstream of the mRNA cap site. TTR proximal promoter region has binding sites for HNF1, HNF3, HNF4, HNF6, and AP-1. Here the target is the proximal region of the promoter to modulate the gene expression.

An approach similar to that described in Example 1 was followed and based upon this analysis three putative sequences were identified for inclusion in gRNAs in order to confer liver specific targeting of the TTR gene as follows:

SEQ ID NO: 3
5′-GACAAGGUUCATAUUUGUAU-3′

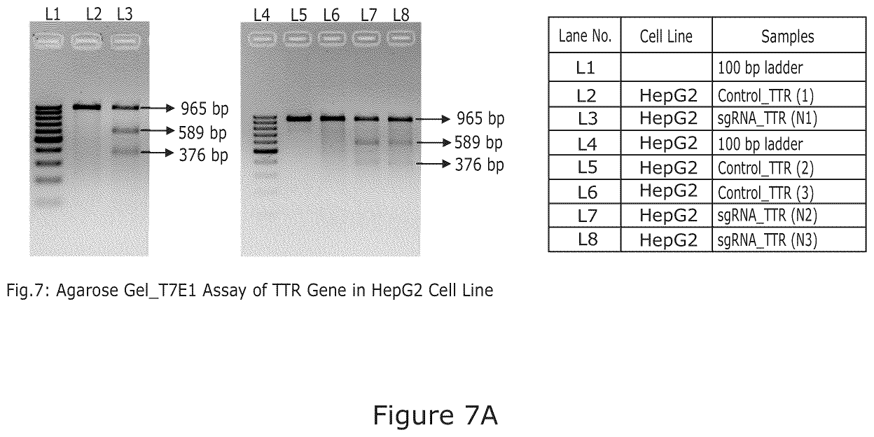

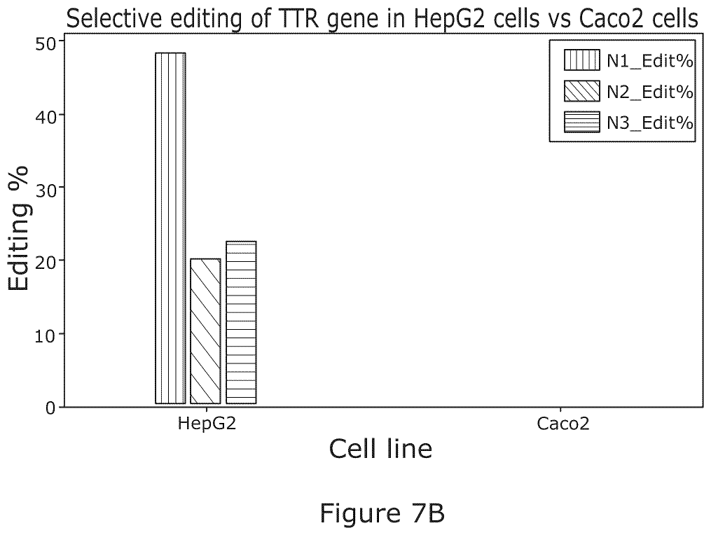

Highly selective editing was shown via T7E1 assay in a comparison between HepG2 (representative of liver cells) as the target cell line to display tissue specificity, and Caco 2 non-target colon cells (see FIGS. 7A and B). The results support the determination of the gRNAs as highly selective to the TTR gene in liver.

### Example 3

Identification of Unique Intronic Liver Hepatocyte Specific Sequences For Use in qRNA Synthesis to Preferentially Target the Liver Gene ANGPTL3

Loss-of-function mutations in Angiopoietin-like 3 (ANGPTL3) are associated with lowered blood lipid levels, making this gene an attractive therapeutic target by gene editing for the treatment of human lipoprotein metabolism disorders.

The hyper target selected is the enhancer of ANGPTL3 which resides in the intronic region of DOCK7 regulating the expression of the gene. Two regions of enhancer chr1:63,049,440-63,091,060 and Chr1:63,074,620-63,074,894 were explored to design gRNAs as per the approach described in Example 1. The regions mentioned showed high expression and accessibility in liver cells (HepG2). Caco2 (representative of colon cells) with low expression and accessibility was selected as the comparator non-target tissue. One putative sequences was identified for inclusion in gRNAs in order to confer liver specific targeting of the TTR gene as follows:

SEQ ID NO: 4
5′-AGCAGUGUGUUUGGAGAGCA-3′

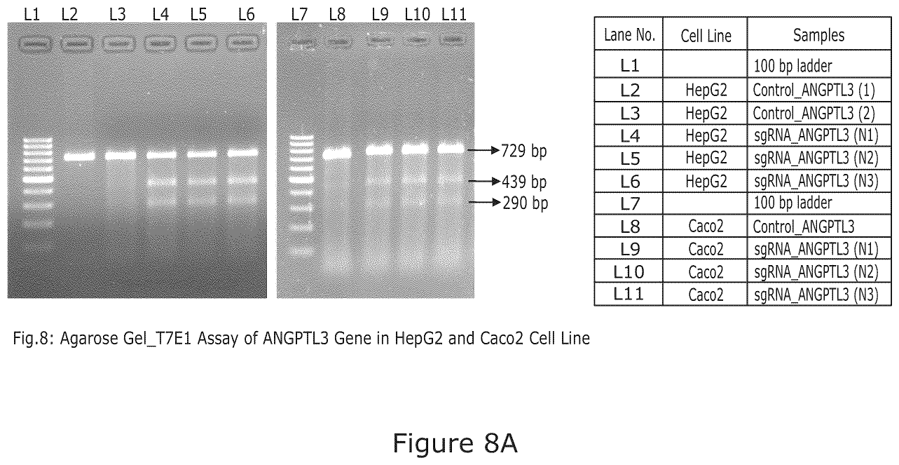

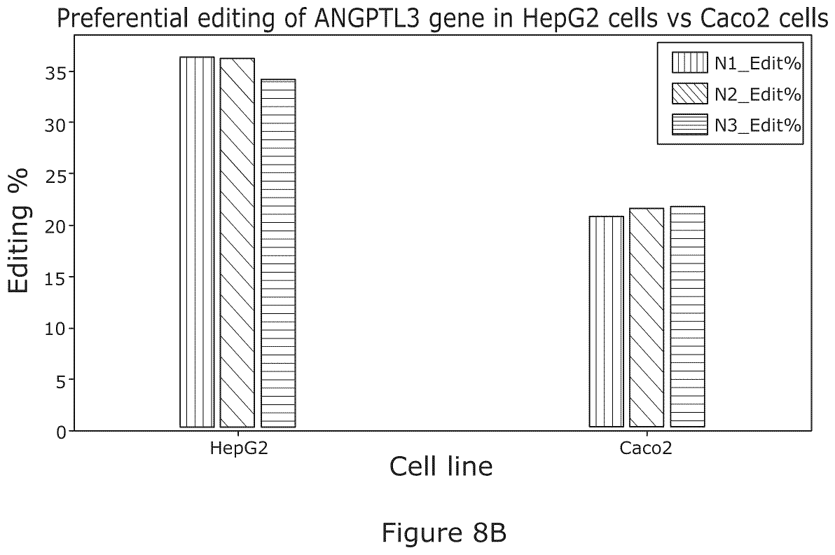

More preferential editing was shown via T7E1 assay in a comparison between HepG2 (representative of liver cells) as the target cell line to display tissue specificity, and Caco 2 non-target colon cells (see FIGS. 8A and B). The results support the determination of the gRNAs as more preferential to the ANGPTL3 gene in liver than in non-target tissue. Whilst preferential editing is less definitive than highly selective editing it also offers a huge benefit in terms of extremely low off-target editing in the non-target cells/tissues in comparison to the target cells/tissues. This ensures that there is minimal impact thereby adding further to safety. Identification of gRNA sequences with a preferential editing property can be combined with other known vector targeting strategies (e.g. targeted LNPs or viral vectors) to even improve further the tissue selectivity of a resultant gene editing therapy.

### Example 4

### Identification of Unique Exonic Liver Hepatocyte Specific Sequences For Use in Grna Synthesis to Preferentially Target the Liver Gene KLKB1

KLKB1, or plasma kallikrein, plays a crucial role in the pathogenesis of hereditary angioedema (HAE). HAE is a genetic disorder characterized by recurrent episodes of debilitating and potentially fatal swelling in various body tissues, including the skin, gastrointestinal tract, face, hands and respiratory system. In HAE, a deficiency or dysfunction of C1 inhibitor (C1-INH), a protein that regulates the activity of plasma kallikrein, leads to excessive activation of the kallikrein-kinin system. Plasma kallikrein is responsible for the cleavage of high-molecular-weight kininogen (HK), resulting in the release of bradykinin, a potent vasodilator and mediator of inflammation. Excessive bradykinin production leads to increased vascular permeability, oedema formation, and inflammation, characteristic features of HAE.

Targeting plasma kallikrein as a therapeutic approach in HAE aims to inhibit the excessive bradykinin production and thereby prevent angioedema attacks. Several strategies have been developed to target plasma kallikrein, including monoclonal antibodies and small molecules. These agents inhibit the enzymatic activity of plasma kallikrein, reducing bradykinin generation and subsequent symptoms associated with HAE. Inhibiting plasma kallikrein has shown promising results in the management of HAE. By blocking bradykinin production, these therapies can effectively prevent or reduce the frequency and severity of angioedema attacks in HAE patients.

Hence, the development of more targeted therapies against the KLKB1 gene provides an important treatment option for individuals with HAE, offering improved quality of life and reducing the risk of potentially life-threatening complications associated with the condition.

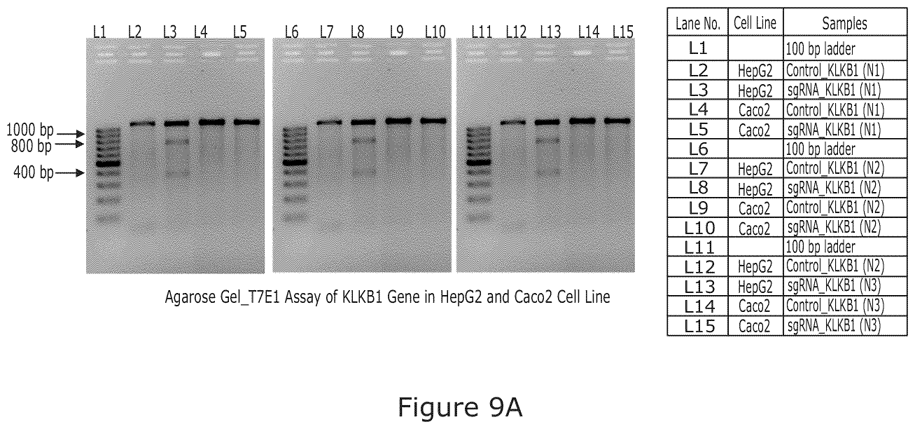

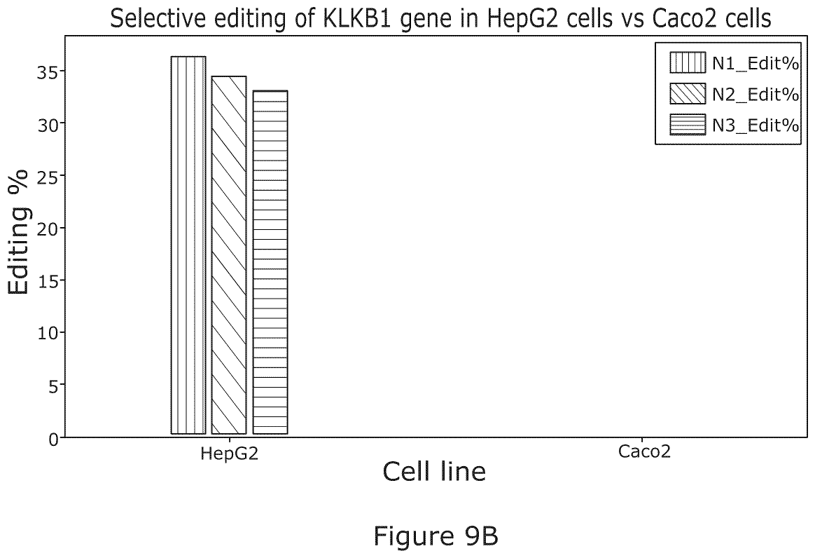

The expression of KLKB1 is higher in HepG2 (representative of liver cells and target cell line) compared to Caco2 (representative of colon and non-target cell line). A guide RNA was designed according to the approaches described herein on the basis of expression and accessibility in the target and non-target cell line. The target region selected wasin an exonic region of chromosome 4 which was demonstrated to display highly selective editing in HepG2. Highly selective editing weas indeed shown via T7E1 assay in a comparison between HepG2 (representative of liver cells) as the target cell line to display tissue specificity, and Caco 2 non-target colon cells (see FIGS. 9A and B).

### Material and Methods

General materials—sgRNAs were procured from Synthego; TrueCut Cas9 Protein v2, Lipofectamine CRISPRMAX, DMEM, OptiMEM, FBS (fetal bovine serum) were procured from ThermoFisher Scientific. Alt-R Genome Editing Detection Kit was procured from IDT. Immortalised human cell lines were procured from ATCC and NCCS, Pune, India.

sgRNA and Cas9 protein complexation—Working stock solutions of Cas9 protein and sgRNA in OptiMEM media were mixed together along with Lipofectamine Cas9 plus reagent. The mixture is incubated for 5 minutes at room temperature to allow the Cas9/sgRNA complex self-assembly. The mole ratio of Cas9 protein to sgRNA used was 1:3.

In vitro transfection—The transfection of guide RNA and Cas9 protein (RNP complex) with Lipofectamine CRISPRMAX was done in HepG2 (representative of liver), A549 (representative of lung) and Caco2 (representative of colon) cell lines. The HepG2, Caco2 and A549 cells were seeded at a concentration of 75,000 and 50,000 respectively in a 24 well plate and allowed to grow for 24 hours in their respective growth medium. After 24 hours, cells were washed with 1× PBS and the RNP complex was subsequently delivered into cultured cells with the help of transfection solution (Lipofectamine CRISPRMAX). Transfected cells were incubated for 48 hours under standard growth conditions of 5% CO2 and 37° C. Post incubation, cells were trypsinized and proceeded for kit based genomic DNA isolation. On-target and off target edits were confirmed by sanger sequencing and T7E1 assay.

PCR and T7E1 genome editing detection assay—Targeted genomic loci were amplified by PCR with gene specific primers and PCR amplification conditions (mentioned in subsequent sections). The amplified product was subjected to T7E1 assay using Alt-R Genome Editing Detection Kit according to the manufacturer's protocol.

Although particular embodiments of the invention have been disclosed herein in detail, this has been done by way of example and for the purposes of illustration only. The aforementioned embodiments are not intended to be limiting with respect to the scope of the appended claims, which follow. The choice of nucleic acid starting material, the clone of interest, or type of library used is believed to be a routine matter for the person of skill in the art with knowledge of the presently described embodiments. It is contemplated by the inventors that various substitutions, alterations, and modifications may be made to the invention without departing from the spirit and scope of the invention as defined by the claims.

What is claimed is:

1. A method for making a guide RNA (gRNA), wherein the gRNA comprises a nucleic acid sequence that is configured to hybridise with a target nucleic acid sequence within the genome of a cell, wherein the target nucleic acid sequence is characterised as being comprised within a locus that is active within a specific cell type, the method comprising the steps of:
identifying one or more candidate nucleic acid sequences that are unique to a specific cell type as compared to a control cell that is selected from a different cell type, thereby identifying tissue specific candidate nucleic acid sequences;
identifying a subset of the candidate nucleic acid sequences of (i) that are comprised within an accessible chromatin region of the genome within the specific cell type; and
iii. synthesising a gRNA that hybridises to one or more of the tissue specific candidate nucleic acid sequences identified in (ii).

2. The method of claim 1, wherein the one or more candidate nucleic acid sequences comprise or are adjacent to a protospacer adjacent motif (PAM) sequence.

3. The method of claim 1, wherein the accessible chromatin region of the genome is comprised within a region of euchromatin.

4. The method of claim 1, wherein the accessible chromatin region of the genome is comprised within a gene.

5. The method of claim 4, wherein the gene is predominantly expressed or uniquely regulated only within the specific cell type.

6. The method of claim 5, wherein the accessible chromatin region of the genome is fully or partially comprised within an untranslated region of the gene.

7. The method of claim 1, wherein the tissue specific candidate nucleic acid sequences are defined as comprising at least one tissue specific gene expression control sequence.

8. The method of claim 7, wherein at least one tissue specific gene expression control sequence is selected from the group consisting of: a promoter; an enhancer; a silencer; an insulator; an miRNA; an IncRNA; a transcription factor; and a transcription factor binding sequence.

9. The method of claim 1, wherein the specific cell type is selected from the group consisting of: muscle; liver; central nervous system (CNS); brain; breast; endothelium; pancreas; esophagus; colon; gastrointestinal organs; kidney; lung; spleen; skin; heart; thyroid; lymphatic tissue; cardiovascular; eye; bone marrow; blood; connective tissue; bladder; reproductive organs; and placenta.

10. The method of claim 9, wherein the specific cell type comprises a diseased cell type.

11. The method of claim 10, wherein the diseased cell type is caused by an intracellular pathogen.

12. The method of claim 10, wherein the diseased cell type is selected from a pre-neoplastic or a neoplastic cell type and wherein neoplastic cell type is selected from the group consisting of: a primary tumour cell; a secondary tumour cell; a metastatic tumour cell; and a cancer stem cell.

13. The method of claim 1, wherein the gRNA is a single gRNA (sgRNA).

14. The method of claim 1 wherein the gRNA is selected based on optimal on-target cleavage and minimum off-target activity predictions.

15. A nucleic acid library that comprises a plurality of nucleic acid sequences that encode a plurality of gRNAs identified via the method of claim 1.

16. An engineered guide RNA (gRNA) capable of forming a complex with a CRISPR-Cas effector protein and directing the CRISPR-Cas complex to a tissue or cell type specific target sequence within a locus of a cell; wherein the gRNA comprises a sequence that hybridises with the tissue or cell type specific target sequence, and wherein the gRNA synthesised by a method as described in claim 1.

17. A CRISPR-Cas complex that comprises an engineered guide RNA (gRNA) in a complex with a CRISPR-Cas endonuclease protein, wherein the gRNA is capable of directing the complex to a tissue or ceil type specific target sequence within a locus of a cell; wherein the gRNA comprises a sequence that hybridises with the tissue or cell type specific target sequence, and wherein the gRNA is synthesised by the method of claim 1.

18. The CRISPR-Cas complex of claim 17, wherein the CRISPR-Cas endonuclease is selected from the group consisting of: Cas9; Cpf1; c2cl; C2c2; Casl3; c2c3; Cas1; Cas1B; Cas2; Cas3; Cas4; Cas5; Cas5e (CasD); Cas6; Cas6e; Cas6f; Cas7; Cas8; Cas8a; CasBal; Cas8a2; Cas8b; Cas8c; Csnl; Csx12; Cas9; Cas10; Cas10d; Cas12a; Cas12b; Cas12c; Cas12d; Cas12e; Cas13a; Cas13b; Cas13c; Cas13d; CasF; CasG; CasH; Csyl; Csy2; Csy3; Csel (CasA); Cse2 (CasB); Cse3 (CasE); Cse4 (CasC); Cse5; Cscl; Csc2; Csa5; Csn2; Csm2; Csm3; Csm4; Csm5; Csm6; Cmrl; Cmr3; Cmr4; Cmr5; Cmr6; Csbl; Csb2; Csb3; Csxl7; Csxl4; CsxIO; Csxl6; CsaX; Csx3; Csxl; Csxl5; Csfl; Csf2; Csf3; Csf4; and Cul966, or a derivative thereof; a variant thereof; and a fragment thereof.

19. The CRISPR-Cas complex of claim 18, wherein the CRISPR.Cas endonuclease is Cas9 or a derivative thereof; a variant thereof; and a fragment thereof.

20. The CRISPR-Cas complex of claim 18, wherein the CRISPR-Cas endonuclease is Cpf1 or a derivative thereof; a variant thereof; and a fragment thereof.
